(19) United States
(12) Patent Application Publication
(10) Pub. No.: US 2013/0302362 A1
(43) Pub. Date: Nov. 14, 2013

(54) Immunomodulatory Compositions, Methods and Systems Comprising Immunogenic Fragments of ApoB-100

(71) Applicants: Prediman K. Shah, Los Angeles, CA (US); Kuang-Yuh Chyu, Los Angeles, CA (US)

(72) Inventors: Prediman K. Shah, Los Angeles, CA (US); Kuang-Yuh Chyu, Los Angeles, CA (US)

(73) Assignee: CEDARS-SINAI MEDICAL CENTER, Los Angeles, CA (US)

(21) Appl. No.: 13/884,916

(22) PCT Filed: Nov. 11, 2011
(86) PCT No.: PCT/US11/60483

§ 371 Date: May 10, 2013

Related U.S. Application Data
(60) Provisional application No. 61/413,378, filed on Nov. 12, 2010.

(51) Int. Cl. C07K 7/08

(52) U.S. Cl. CPC C07K 7/08
USPC 424/185.1

(57) ABSTRACT

Immunostimulatory agents, T cell, compositions, methods and systems for treating and/or preventing various conditions in a human individual.

### CROSS REFERENCE TO RELATED APPLICATIONS

The present application claims priority to U.S. Provisional Application Ser. No. 61/413,378 entitled “Immunomodulatory Compositions, Methods and Systems Comprising Immunogenic Fragments of Apob100” filed on Nov. 12, 2010, with docket number P700-USP, which is herein incorporated by reference in its entirety. The present application is also related to PCT application WO 02/080954 filed on Apr. 5, 2002, to PCT application Ser. No. ______ entitled “Immunomodulatory Methods and Systems for Treatment and/or Prevention of Aneurysms” filed on Nov. 11, 2011 with docket number P686-PCT, and to PCT application Ser. No. ______ entitled “Immunomodulatory Methods and Systems for Treatment and/or Prevention of Hypertension” filed on Nov. 11, 2011 with attorney docket P694-PCT, each of which is herein incorporated by reference in its entirety.

### FIELD

The present disclosure relates to immunomodulatory compositions methods, systems, compositions, and in particular vaccines that comprise immunogenic fragments of ApoB100 are suitable for the treatment or prevention of conditions such as atherosclerosis, aneurysms, hypertension and/or of a condition associated thereto.

### BACKGROUND

Immunogenic fragments of ApoB100 have been associated with treatment and/or prevention to various conditions in an individual.

In particular, certain immunogenic fragments of ApoB100 have been associated with treatment of atherosclerosis in individuals.

### SUMMARY

Provided herein are compositions, methods and systems that allow in several embodiments treatment and/or prevention of various conditions treated with immunogenic fragments of ApoB100 in a human individual.

According to a first aspect, a method to treat and/or prevent a condition in a human individual, is described the condition treatable and/or preventable by administering one or more immunogenic fragments of ApoB100 or an immunogenically active portion thereof. The method comprises administering to an individual the one or more immunogenic fragment of apoB-100 or the immunogenically active portion thereof at a concentration of less than 1 mg.

According to a second aspect, a pharmaceutical composition comprising less than 1 mg of one or more immunogenic fragments of ApoB100 or an immunogenically active portion thereof and a pharmaceutically acceptable vehicle.

The compositions, methods and systems herein described can be used in connection with applications wherein treatment of a condition treatable and/or preventable by administering one or more immunogenic fragments of ApoB100 or an immunogenically active portion thereof in a human individual is desired.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and the examples, serve to explain the principles and implementations of the disclosure.

FIG. 1 shows a representation of the locations of the segments examined for average diameter of segmental aneurysm according to an embodiment herein described.

FIG. 2 shows a Kaplan Meier survival curve for mice immunized with or without p210 according to an embodiment herein described.

FIG. 3 shows p210 immunization confers athero-protective effect. (A) Immunization with native p210 resulted in a significant reduction in aortic atherosclerosis when compared to PBS and cBSA/Alum group (n=9-10 each group, representative picture from each group shown). (B) P210 immunization significantly reduced macrophage infiltration and DC presence assessed by MOMA-2 (n=9-10 each group) and CD11c (n=7-12 each group) immuno-reactivity, respectively in aortic sinus plaques.

FIG. 4 Effect of p210 immunization on DCs. One week after primary immunization, (A) CD11c(+) or (B) CD11c(+)CD86(+) cells at the immunization sites was significantly reduced in p210/cBSA/alum group when compared to cBSA/alum group. N=10 each group. (C) One week after third immunization, p210 immunized mice had reduced CD11c(+)CD86(+) cells in lymph nodes compared to cBSA/alum group (n=5 in each group; ANOVA followed by multiple group comparison).

FIG. 5 shows IgM or IgG titer against p210 before and after p210 immunization. (A) The p210 IgG titers were low before immunization and remained low in the PBS group at euthanasia but significantly increased in cBSA/alum and p210/cBSA/alum groups, with the highest titer in the cBSA/alum group. (B) The p210 IgM titers were low before immunization and significantly increased at euthanasia with no difference among 3 groups of mice. N=5 for 6-7 week time-point and n=9 for 25 week time-point.

FIG. 6 shows activated lymphocyte population after immunization in vivo. (A) CD8(+)CD25(+) T-cell population in the lymph nodes was significantly higher in p210/cBSA/alum group when compared to that of PBS or cBSA/alum groups; (B) CD4(+)CD25(+) T-cells in the lymph nodes did not differ among the three groups. There was a significantly larger population of splenic CD8(+)CD25(+)IL-10(+) T-cells in p210/cBSA/alum group among 3 groups (C) without difference in splenic CD8(+)CD25(+)IL12(+) T-cells among 3 groups (D). Splenic CD4(+)CD25(+)IL-10(+) T-cell population significantly increased in the cBSA/alum group, but was significantly attenuated by the p210/cBSA/alum immunization (E) and (F) splenic CD4(+)CD25(+)IL12(+) T-cells did not differ among 3 groups. N=9-10 in each group for (A) and (B); n=5 in each group for (C), (D), (E) and (F).

FIG. 7 shows adoptive transfer of CD8(+) T-cells from p210 immunized donors recapitulated the athero-protective effect of p210 immunization but not by transfer of B-cells or CD4(+)CD25(+) T-cells. (A) The recipient mice of CD8(+) T-cells from p210/cBSA/alum immunized donors developed significantly smaller atherosclerotic lesions compared to the recipient mice of CD8(+) T-cells from other 2 groups (n=9-10 each group). (B) Adoptive transfer of B-cells from p210/cBSA/alum donors did not reduce atherosclerosis when compared to the recipient mice of B-cells from PBS or cBSA/alum groups (n=9 each group). Recipient mice of CD4(+)CD25(+) T-cells (n=9-13 each group) with 2 different doses (C. 1×10⁵ cells/mouse or D. 3×10⁵ cells/mouse) did not reproduce the athero-reducing effect of p210 immunization.

FIG. 8 shows increased cytolytic activity of CD8(+) T cells from p210 immunized mice against dendritic cells in vitro. CD8(+) T-cells from p210 immunized mice significantly had a higher cytolytic activity against dendritic cells when compared to those from PBS or BSA/alum groups. Experiments were repeated 4 times with CD8(+) T-cells pooled from 5 mice in each group each time. Duplicate or triplicate was done each time with total of 11 data-points in each group altogether.

FIG. 9 shows CD8(+) T-cells from p210 immunized mice containing higher level of Granzyme B when compared to those from PBS or cBSA/alum group; whereas there is no difference in perforin level.

FIG. 10 shows IgG titers against KLH or TNP after p210 immunization. (A) Prior immunization with p210 did not affect the efficacy of subsequent T-cell dependent (KLH, n=3-6 each group) or (B) T-cell independent (TNP, n=4-5 each group) immunization as assessed by the IgG antibody titers when compared to mice received PBS or cBSA/alum.

FIG. 11 shows the effect of p210 immunization on mean blood pressure in various groups of mice according to an embodiment herein described.

FIGS. 12A and 12B shows the effect of p210 immunization on heart rate and mean blood pressure in various groups of mice according to embodiments herein described.

FIG. 13 shows a Kaplan Meier survival curve for mice immunized with or without p210 according to one embodiment herein described.

FIG. 14 shows Antibody response to p210 in apoE−/− mice according one embodiment herein described.

FIG. 15 shows cytolytic activity of p210-immune CD8⁺ T cells is abrogated by depletion of CD25⁺ cells. Lytic activity specific to p210 is also abrogated by absence of serum lipids in the assay medium.

FIG. 16 shows endocytosis of FITC-labeled p210 by DCs according one embodiment herein described.

FIG. 17 shows presentation of the peptide p210 by DCs to CD8⁺ CD25⁻ T cells in vitro as shown by increased activated CD25+ cells according one embodiment herein described.

FIG. 18 shows CD8⁺ lytic activity gated on FITC cells according an embodiment herein described. p210-specific lytic activity by CD8⁺ T cells from p210-vaccinated mice using DCs loaded with FITC-labeled p210.

### DETAILED DESCRIPTION

Methods and systems are herein described that allow in several embodiments, treatment and/or prevention of various conditions in a human individual.

The term “treat,” or “treating” or “treatment” as used herein indicates any activity that is part of a medical care for, or that deals with, a condition medically or surgically. The term “preventing” or “prevention” as used herein indicates any activity, which reduces the burden of mortality or morbidity from a condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “condition” as used herein indicates the physical status of the body of an individual (as a whole or of one or more of its parts) that does not conform to a physical status of the individual (as a whole or of one or more of its parts) that is associated with a state of complete physical, mental and possibly social well-being. Conditions herein described include but are not limited to disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms. Exemplary conditions include but are not limited to injuries, disabilities, disorders (including mental and physical disorders), syndromes, infections, deviant behaviours of the individual and atypical variations of structure and functions of the body of an individual or parts thereof.

In some embodiments, treatment and/or prevention of various conditions can be provided by administering to a human individual an effective amount of one or more immunogenic fragments of ApoB100 or an immunogenically active portion thereof, wherein the effective amount is less than 1 mg.

The term “administer” or “administering” or “administration” as used herein means any method of providing an individual with a substance in any fashion including, but not limited to, those discussed herein.

The term “individual” or “individuals” as used herein indicates a single biological organism such as higher animals and in particular vertebrates such as mammals and more particularly human beings. In some embodiments, the individual has been previously identified as having an increased risk of aneurysm based on the detection of conditions typically associated with an increased risk of aneurysm (e.g. higher blood pressure, atherosclerosis). In some embodiments, the individual has not been identified as having an increased risk of aneurysm. In some embodiments, no investigation as to the risk for aneurysm in the individual has been performed.

The term “immunogenic fragment” or “antigenic fragment” as used herein indicates a portion of a polypeptide of any length capable of generating an immune response, such as an antigen. An antigen is a molecule recognized by the immune system. An antigenic fragment of ApoB100 is accordingly a portion of ApoB-100 that presents antigenic properties. The ability of a fragment or other molecule to generate an immune response and in particular a cellular and/or humoral response can be detected with techniques and procedures identifiable by a skilled person.

The term fragment in the sense of the present disclosure comprises not only fragments of any length from ApoB100, but also peptides produced by genetic recombination or chemically synthesized. The term “immunogenic fragments” in the sense of the present disclosure further comprise also derivative of any fragment, such as oxidative derivative and/or peptide treated with MDA or copper, which maintain a detectable antigenic property of the original fragment.

The term “derivative” as used herein with reference to a first peptide (e.g., an immunogenic fragment), indicates a second peptide that is structurally related to the first polypeptide and is derivable from the first polypeptide by a modification that introduces a feature that is not present in the first peptide while retaining functional properties of the first peptide. Accordingly, a derivative peptide of an immunogenic fragment, or of any portion thereof, e.g. an epitope thereof, usually differs from the original an immunogenic fragment or portion thereof by modification of the amino acidic sequence that might or might not be associated with an additional function not present in the original peptide or portion thereof. A derivative peptide of an immunogenic fragment or of any portion thereof retains however one or more of the immunogenic activities that are herein described in connection with an immunogenic fragment or portion thereof. The antigenic properties can be verified with methods and systems such as the ones already described for the immunogenic fragments and additional methods and systems identifiable to a skilled person. Typically, a derivative of an immunogenic fragment comprises at least one epitope of the immunogenic fragment.

The term immunogenically active portion in the sense of the present disclosure indicates any part of a reference antigen that can elicit specific immune response. Exemplary immunogenically active portions are the epitopes formed by 5 or more residues comprised within an immunogenic fragment. In some embodiments, epitopes within one fragment can overlap.

Immunogenic fragments can be expressed by recombinant technology, such as a fusion with an affinity or epitope tag, chemical synthesis of an oligopeptide, either free or conjugated to carrier proteins, or any other methods known in the art to express the ApoB-100 peptides.

Exemplary fragments of ApoB100 are peptides each comprising one of the sequences listed in the Sequence Listing as SEQ ID NO: 1 to SEQ ID NO: 302 described in further detail in the Examples section. Methods and systems suitable to identify an immunogenic fragment in the sense of the present are described in WO 02/080954, hereby incorporated by reference. Additional methods are exemplified in the Examples section (see e.g. Example 1). The term “protein” or “polypeptide” or “peptide” as used herein indicates an organic polymer composed of two or more amino acid monomers and/or analogs thereof. The term “polypeptide” includes amino acid polymers of any length including full length proteins and peptides, as well as analogs and fragments thereof. A polypeptide of three or more amino acids is also called an oligopeptide. As used herein the term “amino acid”, “amino acidic monomer”, or “amino acid residue” refers to any of the twenty naturally occurring amino acids including synthetic amino acids with unnatural side chains and including both D and L optical isomers. The term “amino acid analog” refers to an amino acid in which one or more individual atoms have been replaced, either with a different atom, isotope, or with a different functional group but is otherwise identical to its natural amino acid analog. In some embodiments, the one or more immunogenic fragments of ApoB100 is associated to atherosclerosis reduction.

Methods to identify a molecule associated with atherosclerosis reduction are identifiable by a skilled person and include the exemplary procedures described in WO 02/080954 herein incorporated by reference in its entirety. In particular, the ability of a molecule to reduce atherosclerosis can be tested in an animal model following administration of the molecule in a suitable amount using procedure identifiable by a skilled person. For example following subcutaneous administration of a molecule herein described the ability of the molecule to affect atherosclerosis can be tested in mice as illustrated in the Examples sections. A skilled person will be able to identify additional procedure, schedule of administration and dosages upon reading of the present disclosure.

Accordingly in an exemplary embodiment, immunogenic molecule associated with atherosclerosis reduction can be identified by identifying a candidate immunogenic molecule able to provide a cellular and/or humoral response in the individual of interest; and testing the candidate immunogenic molecule for an ability to reduce atherosclerosis, to select the candidate immunogenic molecule associated with atherosclerosis reduction.

In particular, in some embodiments, immunogenic fragments of ApoB100 are immunogenic fragments producing an immune response associated to atherosclerosis reduction in the individual or in an animal model. In some of those embodiments, a percentage atherosclerosis reduction is at least about 20%, or at least about 30%, from about 40% to about 60% or about 50% to about 80%.

In some embodiments, the immunogenic fragment comprises at least one of peptide, each comprising p1 (SEQ ID NO: 1), p2 (SEQ ID NO: 2), p 11 (SEQ ID NO:11), p25 (SEQ ID NO:25), p45 (SEQ ID NO:45), p74 (SEQ ID NO:74), p99 (SEQ ID NO:99), p100 (SEQ ID NO:100), p102 (SEQ ID NO:102), p103 (SEQ ID NO: 103), p105 (SEQ ID NO:105), p129 (SEQ ID NO:129), p143 (SEQ ID NO:143), p148 (SEQ ID NO:148), p210 (SEQ ID NO:210), or p301 (SEQ ID NO:301).

In an embodiment, the one or more immunogenic fragments comprise one or more peptides each comprising p2 (SEQ ID NO:2), p 11 (SEQ ID NO:11), p45 (SEQ ID NO: 45), p74 (SEQ ID NO: 74), p102 (SEQ ID NO: 102), p148 (SEQ ID NO:148), or p210 (SEQ ID NO:210).

In an embodiment, the one or more immunogenic fragments comprise two peptides each comprising p143 (SEQ ID NO: 143), or p210 (SEQ ID NO:210). In an embodiment, the one or more immunogenic fragments associated to atherosclerosis reduction comprises three peptides each comprising, one of p 11 (SEQ ID NO:11), p25 (SEQ ID NO: 25), or p74 (SEQ ID NO:74). In an embodiment, the one or more immunogenic fragments associated to atherosclerosis reduction comprises five peptides each comprising one of p99 (SEQ ID NO: 99), p100 (SEQ ID NO: 100), p102 (SEQ ID NO: 102), p103 (SEQ ID NO: 103), and p105 (SEQ ID NO: 105).

In an embodiment, the one or more immunogenic fragments comprise one or more peptides each comprising p2 (SEQ ID NO: 2), p45 (SEQ ID NO: 45), p74 (SEQ ID NO: 74), p102 (SEQ ID NO: 102), or p210 (SEQ ID NO:210).

In an embodiment, the one or more immunogenic fragments comprise a peptide comprising amino acids 16-35 of human apoB-100 (p2; SEQ ID NO:2).

In an embodiment the one or more immunogenic fragments comprise a peptide comprising amino acids 661-680 of human apoB-100 (p45; SEQ ID NO:45).

In an embodiment, the one or more immunogenic fragments comprise a peptide comprising amino acids 3136-3155 of human apoB-100 (P210; SEQ ID NO: 210).

In an embodiment, the one or more immunogenic fragments comprise a peptide comprising amino acids 4502-4521 of human apoB-100 (P301; SEQ ID NO: 301).

In an embodiment, the one or more immunogenic fragments comprise a peptide comprising amino acids 1-20 of human apoB-100 (P1; SEQ ID NO: 1).

Exemplary data showing association of the above peptides to atherosclerosis reduction are shown in Example 5 of the present disclosure and in International application WO 02/080954, herein incorporated by reference in its entirety (see in particular Table 1, Table 2, Table A and Table B). In particular for some of those peptides or combination thereof a percentage reduction of 64.6% (p143 and p210), 59.6% (p11, p25 and p174), 56.8% (p129, p148, and p167), p67.7 (p2), 57.9% (p210), 55.2% (p301), 47.4% (p45), 31% (p1) has been detected (see W0/02080954 incorporated herein by reference in its entirety, and in particular Table B).

Immunogenic peptides comprising any of the sequences herein described or immunogenically active portions of those peptides are identifiable by a skilled person using in silico and/or in vitro approaches. For example, in silico methods can be used to identify any of said epitopes or immunogenic peptides based on any of the sequences herein described. Reference is made for example, to the papers [44] to [51] each of which are incorporated herein by reference in its entirety.

Such papers describe various algorithms such as Tepitope (Radrizzani et al 2000), Adept (Maksuytov et al 1993), antigenic index (Jameson et al 1988) and others which can be used to identify the immunogenic molecules comprising the sequences at issue or any relevant epitopes.

Additional tests and laboratory procedures in vitro and/or in vivo suitable to be used alone or in connection with the identification in silico (e.g. ELISA, antigen-specific T cell proliferation assay, ELISPOT, antibody measurement) are identifiable by a skilled person that can be used by a skilled person to verify the in silico data and/or identify immunologically active molecules comprising any of the sequences herein described or immunologically active portions of those sequences.

Accordingly, in an exemplary embodiments, immunogenic peptides, herein described, immunogenically active portions thereof as well as derivative thereof can be identified by identifying candidate peptides, candidate active portion and/or candidate derivative by in silico analysis of any one of the sequences herein described, and by identifying the immunogenic peptides, immunogenically active portions and/or derivative by in vitro and/or in vivo testing of the candidate peptides, candidate active portion and/or candidate derivative. In particular, the in silico analysis can be performed by analyzing the sequence of the candidate with algorithm suitable to identify immunogenicity of a molecule or portion thereof. Similarly, the in vitro and/or in vivo testing comprises methods directed to identify immunogenicity of the candidate peptide, candidate active portion and/or derivative as well as effects of those molecules on aneurysm, with particular reference to formation or regression. Suitable methods and techniques are identifiable by a skilled person upon reading of the present disclosure.

In several embodiments, the immunogenic peptides, active portions thereof and derivative thereof are expected to include a sequence of at least about 5 amino acids, consistently with the typical length of epitopes as indicated in WO 02/080954 herein incorporated by reference in its entirety.

Additional conditions treatable with immunogenic fragments or an immunogenically active portion thereof herein described comprise aneurysmand hypertension.

The term “aneurysm” as used herein indicates a localized blood filled dilation of a blood vessel or of a portion thereof. In particular, an aneurysm can be an abnormal widening or ballooning of a portion of an artery due to weakness in the wall of the blood vessel, and can occur within any vasculature in the body. Aneurysms can be “true” in which the inner layers of a blood vessel bulges outside the outer layer of the vessel, or “false,” which is a collection of blood leaking out of an artery or vein. Aneurysms commonly occur, but are not limited to, in arteries at the base of the brain or aortic in the main artery carrying blood from the left ventricle of the heart. In particular, with reference to the aorta, aneurysms can occur at different segments of the aorta including, but not limited to, the beginning of the arch, the end of the arch, the apex, between segments 3 and 5, the supra renal segment, the infra renal segment, before bifurcation, and between the renal artery. Symptoms of aneurysms include pain, peripheral embolization, bleeding and additional symptoms identifiable by a skilled person.

In an embodiment, immunization with one or more of the immunogenic molecules herein described reduces the incidence of experiencing aortic aneurysm rupture (e.g. Examples 2 and 11).

In an embodiment, immunization with one or more of the immunogenic molecules herein described reduces the aortic aneurysmal segment formation. In particular, some of those embodiments, reduction of aneurysms can occur at different segments of the aorta including, but not limited to, the beginning of the arch, the end of the arch, the apex, between segments 3 and 5, the supra renal segment, the infra renal segment, before bifurcation, and between the renal arteries (se e.g. Example 3). The expected reduction of aneurysm after immunization is at least about 20%, and in particular about 20-80% when compared to a control measurement.

In an embodiment, immunization with one or more of the immunogenic molecules herein described reduces mortality associated with aortic aneurysmal rupture (see e.g. Examples 4 and 11).

In an embodiment, immunization with one or more of the immunogenic molecules herein described is associated with a reduction in hypertension.

The term “hypertension” as used herein refers to high blood pressure. In particular, hypertension (HTN) or high blood pressure is a chronic medical condition in which the systemic arterial blood pressure is elevated. It is the opposite of hypotension. It is classified as either primary (essential) or secondary. About 90-95% of cases are termed “primary hypertension”, which refers to high blood pressure for which no medical cause can be found. The remaining 5-10% of cases (Secondary hypertension) is caused by other conditions that affect the kidneys, arteries, heart, or endocrine system.

In an embodiment, immunization with one or more of the immunogenic molecules herein described reduces the incidence of blood pressure (e.g. Examples 10 and 11).

The expected reduction of blood pressure after immunization is at least about 10%, when compared to a control measurement and in particular from about 10% to an amount determined by a physician based on the condition and the individual to be immunized (e.g. Examples 10 and 11).

The term “effective amount” as used herein is meant to describe that amount of antigen, e.g. p210, which induces an antigen-specific immune response.

Effective amounts of an immunogenic fragment and of one or more of the immunogenic molecules herein described to treat and/or prevent a condition will depend on the individual wherein the activation is performed and will be identifiable by a skilled person upon reading of the present disclosure For example in an embodiment a desired effect can be achieved in mice with an effective amount of from about 100 μg to less than about 1000 μg immunogenic fragment or immunogenically active portion thereof.

It has now unexpectedly found that treatment of human individuals with concentration similar to the ones used in mice are particularly effective for all the applications wherein one or more immunogenic fragments of ApoB100 (which include any derivative thereof see related WO 02/080954, PCT application Ser. No. ______ entitled “Immunomodulatory Methods and Systems for Treatment and/or Prevention of Aneurysms” filed on Nov. 11, 2011 with docket number P686-PCT, and to PCT application Ser. No. ______ entitled “Immunomodulatory Methods and Systems for Treatment and/or Prevention of Hypertension” filed on Nov. 11, 2011 with attorney docket P694-PCT each of which is herein incorporated by reference in its entirety), or an immunogenic portion thereof, are administered to humans.

In an embodiment, an effective amount for the treatment or prevention can be about 100 μg or more. In particular, treatment with about 100 μg is expected to prevent aneurysm rupture in humans (see e.g. Example 2).

A greater concentration can be used in some embodiments depending on the desired effect as illustrated in the present disclosure. For example, in embodiments wherein treatment of a condition is expected to be performed with an effective amount be 250 μg or more and in particular with about 500 μg. In another example, an effective amount to treat the condition is expected to be 250 μg or 500 μg or higher is also expected to be effective also depending on other factors affecting the pharmacological activity of the molecule in an individual.

According to the same data, treatment or prevention of a condition can be performed in humans with an effective amount of from about 0.1 to about 100 mg immunogenic fragment or immunogenically active portion thereof.

In particular, the effective amount is expected to vary depending on the number and combination of peptides utilized for each particular vaccine, and specific characteristic and conditions of the individual treated (e.g. immune system, diet and general health and additional factors identifiable by a skilled person). More particular, lower or higher amounts within the defined range are expected to be effective in an individual depending on factors such as weight, age, gender of the individual as well as additional factors identifiable by a skilled person.

There are several well established methods for extrapolating a human equivalent dose (HED) from a dose that is effective in animals. One popular approach is to convert dose per body weight and another is to use an allometric conversion which takes into account body surface area. These approaches are most reliable for small molecules for which the typical sigmoidal dose-response relationship exists. Dose-response relationships for immune modulating therapies are atypical and often unpredictable. This situation is further complicated by the differences in the immune systems of animals and of humans (see attached). Furthermore, the dose that will work in humans need not be an “n” times greater multiple of the dose that worked in animals. It could be the same or even less. There are some in silico (EpiVax) and in vitro (Vax Design, Probiogen) tools that have recently become available that can be used to more accurately predict what formulations/doses will and will not work in humans.

The effective amount is also expected to vary depending on the number and combination of peptides utilized for each particular vaccine, and specific characteristic and conditions of the individual treated (e.g. immune system, diet and general health and additional factors identifiable by a skilled person). More particular, lower or higher amounts within the defined range are expected to be effective in an individual depending on factors such as weight, age, gender of the individual as well as additional factors identifiable by a skilled person. In some embodiments, the immunogenic peptides herein described or related immunogenically active portions can be administered in combination with an adjuvant or other carrier suitable to affect and in particular increase immunogenicity of the peptide o active portion thereof. In particular, in some embodiments, the immunogenic peptide or active portion thereof can be conjugated to the adjuvant or carrier according to procedures identifiable to a skilled person. Suitable carriers comprise BSA, and in particular, cationized BSA, aluminum salts such as aluminum phosphate and aluminum hydroxide and additional carriers identifiable by a skilled person.

In some embodiments, immunogenic molecules herein described can be administered in ratios of immunogenic molecule to carrier to aluminum of about: 1:2:35, 1:2:20.6, 1:2:7.7, 1:2:3.3, 1:1:13.8 weight to weight ratios. In particular, in some embodiments, ratios can be provided wherein the number of peptides conjugated to each carrier molecule while minimizing the amount of aluminum (adjuvant). In particular in one embodiment, ratio can be provided that result in a concentration up to 2.7 mg conjugate/mL.

The route of immunization can vary depending on the purposes of immunization described herein. For example, successful prevention and treatment of aneurysms in mice occurred by subcutaneous route of administration of immunization (Examples 2, 3, and 4). The type of immune response triggered is largely determined by the route of immunization. Various routes can be used comprising subcutaneous, parenteral, and systemic among the others. In particular, the mucosal linings of airways and intestines contain lymphatic tissue that, when exposed to antigen, elicits anti-inflammatory, immunosuppressive responses. Distinct immunological features of the respiratory and intestinal mucosa lead to partly different types of protective immunity upon antigen exposure by the nasal or oral route.

In some embodiments the immunogenic molecules herein described can be administered according to a schedule of administration devised in view of the amount of time required by the adaptive immune system of an individual to mount a response to the initial exposure to an immunogen. Typically, the response is expected to plateau at 2-3 weeks after exposure. Subsequent exposures often elicit a more rapid response. In various embodiments, the following schedules and manner of administration can be followed: (1) single administration, (2) two administrations 2-3 weeks apart, (3) three weekly administrations, (4) up to 6 administrations on a 1 every 3 week schedule. The vaccines have been administered by: (1) subcutaneous injection; (2) intraperitoneal injection; (3)nasal installation; (4) subcutaneous infusion.

In particular, an embodiment, administering one or more immunogenic fragment or an immunogenically active portion thereof can be performed subcutaneously or intramuscularly.

In some embodiments, disclosed are pharmaceutical compositions which contain at least one the immunogenic fragments, active portions thereof, herein described as herein described, in combination with one or more compatible and pharmaceutically acceptable vehicles, and in particular with pharmaceutically acceptable diluents or excipients. In those pharmaceutical compositions the immunogenic fragments, active portions thereof, herein described can be administered as an active ingredient for treatment or prevention of a condition in an individual.

The term “excipient” as used herein indicates an inactive substance used as a carrier for the active ingredients of a medication. Suitable excipients for the pharmaceutical compositions herein disclosed include any substance that enhances the ability of the body of an individual to absorb an immunogenic fragment, active portions thereof herein described. Suitable excipients also include any substance that can be used to bulk up formulations with the immunogenic fragments, active portions thereof, herein described to allow for convenient and accurate dosage. In addition to their use in the single-dosage quantity, excipients can be used in the manufacturing process to aid in the handling of the immunogenic fragments, active portions thereof, herein described. Depending on the route of administration, and form of medication, different excipients can be used. Exemplary excipients include but are not limited to antiadherents, binders, coatings disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluent include any substance that can decrease the viscosity of a medicinal preparation.

In some embodiments, pharmaceutical composition can include (1) a peptide or other immunogenic molecule herein described administered alone, (2) a peptide or other immunogenic molecule herein described +carrier(s); (3) a peptide or other immunogenic molecule herein described +adjuvant; (4) a peptide or other immunogenic molecule herein described +carrier +adjuvant. In particular, the carriers for each of the exemplary composition (1) to (4) can comprise: (1) cBSA, (2) rHSA, (3) KLH, (4) cholera toxin subunit B, respectively, each of which can be mineral salt-based. Other carriers, known to those skilled in the art, are expected to be suitable as well as will be identified by a skilled person. Examples of those adjuvants comprise adjuvants having Th2 effects, carriers having adjuvant properties, e.g., diphtheria toxoid, and adjuvants able to function as carriers, e.g., oil-water emulsions. In some embodiments, a necessary, and under certain conditions sufficient, component for the pharmaceutical composition is the immunogenic peptides. Additional components of the composition can be selected to modulate the immunological impact of the peptides or other immunogenic molecule herein described as will be understood by a skilled person.

In an embodiment, any of the immunogenic molecules herein described can be used to specifically activate T cell, and in particular CD8(+) T cells which can be activated using one or more immunogenic fragments of ApoB100 or an immunogenically active portion thereof.

The term “T cells” as used herein indicates T lymphocytes belonging to a group of white blood cells known as lymphocytes, and participate in humoral or cell-mediated immunity. T cells can be distinguished from other lymphocyte types, such as B cells and natural killer cells (NK cells) by the presence of special markers on their cell surface such as T cell receptors (TCR). Additional markers identifying T cell include CD1a, CD3, or additional markers possibly associated to a T cell state and/or functionality as will be understood by a skilled person.

The term “CD8(+) T cells” indicates T cells expressing the CD8 glycoprotein at their surface, wherein the CD8 (cluster of differentiation 8) glycoprotein is a transmembrane glycoprotein that serves as a co-receptor for the T cell receptor (TCR). Similarly to the TCR, CD8 binds to a major histocompatibility complex (MHC) molecule, but is specific for the class I MHC protein. Exemplary CD8 T cells comprise cytotoxic memory CD8 T cells, regulatory CD8 T cells, cytotoxic effector CD8 T-cells and additional cells identifiable by a skilled person. There are two isoforms of the protein, alpha and beta, each encoded by a different gene. In humans, both genes are located on chromosome 2 in position 2p12.

The term “activated” and activation as used herein indicate the process by which a T cells interacts with an antigen presenting cell which presents a specific antigen for a time and under condition resulting in a T cell having a preassigned immunological role (e.g. cytotoxicity) within the immune system. The term “antigen-presenting cell” (APC) indicates a cell that displays antigen complex with major histocompatibility complex (MHC) on its surface. T-cells recognize this complex using their T-cell receptor (TCR). Exemplary APCs comprise dendritic cells (DCs) which are known to play an important role in linking innate and acquired immunity, see references (3) (4), and both immune responses participate in atherogenesis, see references (5) (6).

Detection of T cells and in particular, CD8(+) T cells, can be performed by detection of markers such as CD8, alone or in combination with TCR and additional markers identifiable by a skilled person. Detection of activated CD8(+) T cells can be performed by detection of T cells markers and in particular of markers such as CD25, CD44, CD62 and additional markers identifiable by a skilled person using process and techniques suitable for detecting surface markers.

The terms “detect” or “detection” as used herein indicates the determination of the existence, presence or fact of a molecule or cell in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

Exemplary techniques suitable for detecting T cell markers comprise use of suitable monoclonal or polyclonal antibodies or antigen-specific HLA or MHC pentamers or hexamers labeled with an appropriate molecule allowing detection as well as additional methods and techniques identifiable by a skilled person. In an exemplary approach T cell markers are identified by flow cytometric analysis as described in the Examples section. Exemplary techniques suitable for detecting T cell markers comprise use of suitable monoclonal or polyclonal antibodies or antigen-specific HLA or MHC pentamers or hexamers labeled with an appropriate molecule allowing detection as well as additional methods and techniques identifiable by a skilled person. In an exemplary approach T cell markers are identified by flow cytometric analysis as described in the Examples section.

In particular, in some embodiments, an effective amount of immunogenic molecules herein described from about 100 μg to less than about 1000 μg is associated with CD8+ Tcell activation that is specific for the activating immunogenic molecule. (see e.g. Example 12).

Additional effective concentrations of immunogenic fragment or immunogenically active portion thereof for specific CD8+ T cell activation comprise concentration from 100 μg to 250 μg and from 250 μg to about 500 μg and from about 0.1 to about 100 mg.

In particular, T cell activation can be performed using any of the molecules herein described administered in vivo in an amount suitable to treat or prevent aneurysms, (see e.g. Example section). Activation of T cell can also be performed in vitro using methods and procedures such as the ones described in ref [52] as well as additional procedures identifiable by a skilled person. Further advantages and characteristics of the present disclosure will become more apparent hereinafter from the following detailed disclosure by way of illustration only with reference to an experimental section.

In some embodiments, activated CD8(+) T cells herein described are expected to be effective in treatment and/or prevention of a condition treatable with the corresponding activating immunogenic molecule herein described. In particular, in some embodiments, activate CD8(+) T cells herein described are expected to be effective according to a schedule of administration wherein those cells are administered to an individual daily (for up to 21 days) and on an every 10 day schedule (days 0, 10, 20). Additional schedules expected to be effective can be identified by a skilled person based on cell treatments of conditions such as HIV and/or cancer.

Further advantages and characteristics of the present disclosure will become more apparent hereinafter from the following detailed disclosure by way of illustration only with reference to an experimental section.

### EXAMPLES

The compositions, methods system herein described are further illustrated in the following examples, which are provided by way of illustration and are not intended to be limiting.

In particular, the following examples illustrate exemplary immunogenic fragments and methods using fragment p210. A person skilled in the art will appreciate the applicability and the necessary modifications to adapt the features described in detail in the present section, to additional immunogenic fragments, administered subcutaneously or using other routes of administration in vivo or in vitro according to embodiments of the present disclosure.

Unless otherwise indicated the following material and methods were followed in the Examples reported below.

Selection of peptides and their preparation for immunization The establishment and screening of human apoB-100 peptides has been reported (8). Based on Applicants pilot experiments and prior reports, see references (9),(10) Applicants selected peptide 210 (p210, KTTKQ SFDLS VKAQY KKNKH—SEQ ID NO: 210) as a candidate immunogen. Native p210 peptide (Euro-Diagnostica AB, Sweden) was conjugated to cationic bovine serum albumin (cBSA) as carrier using a method described previously see references (3), (4) Alum was used as adjuvant and mixed with peptide/cBSA conjugate with 1:1 ratio in volume. Peptide conjugation and mixing with alum were prepared fresh prior to each immunization.

Immunization Protocols

Male apoE (−/−) mice (Jackson Laboratories) were housed in an animal facility accredited by the American Association of Accreditation of Laboratory Animal Care and kept on a 12-hour day/night cycle with unrestricted access to water and food. The Institutional Animal Care and Use Committee of Cedars-Sinai Medical Center approved the experimental protocols. In a pilot experiment, p210 immunization using 100 μg dose conferred optimum athero-reduction compared to 25 or 50 μg dose. Hence 100 μg dose was used for all subsequent experiments. Mice, maintained on normal chow diet, received subcutaneous primary immunization in the dorsal area between scapulas at 6-7 weeks of age, followed by a booster at 9 and 12 weeks of age. One week after last booster, diet was switched to high cholesterol chow (TD 88137, Harlan-Teklad) and continued until euthanasia at the age of 25 weeks. Separate groups of mice receiving PBS or cBSA/alum at the same immunization time-points served as control. Some mice were sacrificed at 8 or 13 weeks of age to assess immune response against p210.

Tissue Harvesting and Preparation

At euthanasia the hearts were harvested and embedded in OCT compound (Tissue-Tek) for cryo-section. Whole aortas were cleaned, processed and stained with Oil Red 0 to assess the extent of atherosclerosis en face with computer-assisted histomorphometry, see references (3),(4).

Immunohistochemistry and Histomorphometry

The sections from aortic sinus were stained with MOMA-2 (Serotec), or CD11c (eBioscience) antibody to identify macrophages or dendritic cells immunohistochemically using standard protocol. Oil-Red-O stain for plaque size was done using standard protocol. Computer-assisted morphometric analysis was performed to assess histomorphometry as described previously, see references (3),(4).

Serum ELISA

Flat-bottomed 96-well polystyrene plates (MaxiSorp, Germany) were pre-coated with 100 ul (20 μg/ml) p210, KLH, TNP-KLH (Biosearch Technologies T-5060) or BSA (2 μg/ml for IgG or 10 μg/ml for IgM) respectively by incubation overnight at 4° C. to assess antibodies levels using standard protocol. The coating concentration was optimized in pilot experiments. Goat anti-mouse HRP-IgG (Pierce 31437) or IgM (Southern Biotech) were used as detecting antibodies and the bound antibodies were detected by developing in ABTS (Southern Biotech) as substrate and optical density values were recorded at 405 nm.

Flow Cytometric Analysis

Flow cytometric analysis was performed using standard protocols with antibodies listed in Table 1 below and a FACScan (Becton Dickinson) or a CyAn ADP analyzer (Beckman Coulter). For intracellular cytokine staining, Brefeldin A (3 μg/ml) was added to the cultured cells for 2 hours before cells subject to staining procedure. Cell membranes were permeabilized for staining intracellular molecules.

TABLE 1

| Antigen | Clone | Type | Supplier |
|---|---|---|---|
| CD4 | GK1.5 | FITC-Rat IgG2b, κ | BD Pharmingen |
| CD8b.2 | 53-5.8 | FITC-Rat IgG1, κ | BD Pharmingen |
| CD25 | PC61.5 | PE-Rat IgG1, λ | eBioscience |
| IL-10 | JES5-16E3 | Percp-Cy5.5-Rat | eBioscience |
| | | IgG2a, κ | |
| IL-12 | Clone C17.8 | Percp-Cy5.5-Rat | eBioscience |
| | | IgG2b, κ | |
| CD11c | HL3 | FITC-Hamster | BD Pharmingen |
| | | IgG1, λ | |
| CD86 | GL1 | PE-Rat IgG2a, κ | BD Pharmingen |
| TGF-Beta | 1D11 | APC-Mouse IgG1 | R&D system |
| Granzyme B | 16G6 | Alexa-Fluo 647 Rat | eBioscience |
| | | IgG2b, κ | |
| Perforin | eBioOMAK-D | FITC-Rat IgG2a, κ | eBioscience |

Adoptive Transfer Experiment

Male apoE (−/−) mice on regular chow received subcutaneous immunization as described in previous paragraph and were sacrificed at 13 weeks of age as donors. Splenocytes from the same treatment group were pooled before cell isolation. Donor CD8(+) T-cells, CD4(+)CD25(+) T-cells or B-cells were isolated using Dynabeads FlowComp (Invitrogen) according to the manufacturer's protocols. CD4(+) T-cells were negatively selected from the splenocytes followed by positive selection of CD4(+)CD25(+) cells. B cells were negatively isolated whereas CD8(+) T-cells were positively isolated first and released from beads. The purity of pooled CD8(+) T-cells, CD4(+)CD25(+) T-cells and B-cells was 90%, 80% and 70%, respectively. The isolated CD8(+) T-cells (1×10⁶ cells/mouse), CD4(+)CD25(+) T-cells (1×10⁵ or 3×10⁵ cells/mouse) or B-cells (2×10⁷ cells/mouse) were then adoptively transferred to naïve male apoE (−/−) recipient mice at 6-7 weeks of age via tail vein injection. In the published literatures of vascular biology, the number of adoptively transferred lymphocytes varied greatly. For B-cells transfer, the number of 2×10⁷ cells/mouse was chosen based on two prior reports, see references (11),(12). For CD4(+)CD25(+) T-cells transfer, the number of cells transferred ranged from 5×10⁴ cells/mouse to 1×10⁶ cells/mouse in the published literature see references (13),(14),(15). Hence we chose 2 intermediate doses for our experiment. As to CD8(+) T-cells, 1×10⁶ cells was chosen based on a report from the field of autoimmune disease see ref (16). Applicants did not adoptively transferred CD4(+) T-cells because naïve or antigen-primed CD4(+) T-cells are known to be pro-atherogenic see references (17),(18) Recipient mice were fed normal chow until 13 weeks of age when chow was switched to high cholesterol diet until euthanasia at 25 weeks of age. Aortas were harvested to assess the extent of atherosclerosis.

KLH or Trinitrophenyl-Lipopolysaccharide (TNP-LPS) Immunization

Applicants also tested if p210 immunization affected the efficacy of subsequent immunization with other antigens. KLH was chosen as a prototypical T-cell dependent and TNP as a T-cell independent antigen. Male C57/BL6 mice on regular chow received subcutaneous immunization with p210 conjugate or adjuvant control as described in previous paragraphs for apoE (−/−) mice. At 13 and 15 weeks of age mice were subcutaneously immunized with 100 μg KLH (with alum as adjuvant) at injection sites away from p210 sites or injected intraperitoneally with 100 μg TNP-LPS (Sigma). KLH or TNP immunization was done in separate groups of mice. Blood was collected via retro-orbital puncture at euthanasia (16 weeks of age).

In Vitro Generation of BM-Derived Dendritic Cells (BMDCs)

The method for generating BMDC with GM-CSF was adapted from previous publication with modification see reference (19). Briefly, bone marrow cells from femurs and tibiae of male apoE−/− mice were plated into 10 cm culture plates (Falcon) with 20 ml complete RPMI-1640 containing 10 ng/ml GM-CSF (R&D Systems) and 10 ng/ml IL-4 (Invitrogen). Cells were washed and fed on day 3 and day 5 by removing the old medium followed by replenishing with fresh culture medium with GM-CSF and IL-4. On day 8, the immature DC appeared as non-adherent cells under the microscope and harvested by vigorous pipetting and subcultured into new culture plates with 2×10⁵ DCs in 1.5 ml medium.

In Vitro CD8(+) T-Cells Isolation and Co-Culture with Dendritic Cells

Donor mice [male apoE (−/−) mice] for CD8(+) T-cells were immunized with PBS, cBSA/Alum, or cBSA/Alum/P210 according to the schedule described in earlier paragraphs and splenocytes were harvested at 13 weeks of age. CD8(+) T-cells were negatively isolated using a CD8 selection Dynabeads kit (Invitrogen) per manufacturer's protocol. The selected CD8(+) T-cells were then co-culture with DCs in a CD8:DC ratio of 3:1. A series of pilot studies has been performed to determine the optimal CD8:DC ratio for this assay. After co-culture for 4 hours, cells were collected and processed for flow cytometric determination of CD11c and 7-AAD by LSR II flow cytometer (BD Biosciences) and data was analyzed with Summit V4.3 software. Dendritic cell death without CD8(+) T-cells in the co-culture was used as baseline and percentage of specific lysis of cells was calculated using a method described previously, see reference (20).

Statistics

Data are presented as mean+Std. Number of animals in each group is listed in text or description of the figures. Data were analyzed by ANOVA followed by Newman-Keuls multiple group comparison, or by t-test when appropriate. P<0.05 was considered as statistically significant and horizontal bars in each figure indicated statistically significant difference between groups.

### Example 1

### Immunogenic Fragments of ApoB-100

Specific immunogenic epitopes by focusing on the single protein found in LDL, apolipoprotein B-100 (apo B) were characterized. A peptide library comprised of 302 peptides, 20 amino acid residues in length, covering the complete 4563 amino acid sequence of human apo B was produced. The peptides were produced with a 5 amino acid overlap to cover all sequences at break points. Peptides were numbered 1-302 starting at the N-terminal of apo B as indicated in Table 1 below.

TABLE 1

| | | Apolipoprotein | |
|---|---|---|---|
| Peptide | Sequence | B aa | SEQ ID NO |
| P1: | EEEML ENVSL VCPKD ATRFK | aa 1-20 | SEQ ID NO: 1 |
| P2: | ATRFK HLRKY TYNYE AESSS | aa 16-35 | SEQ ID NO: 2 |
| P3: | AESSS GVPGT ADSRS ATRIN | aa 31-50 | SEQ ID NO: 3 |
| P4: | ATRIN CKVEL EVPQL CSFIL | aa 46-65 | SEQ ID NO: 4 |
| P5: | CSFIL KTSQC TLKEV YGFNP | aa 61-80 | SEQ ID NO: 5 |
| P6: | YGFNP EGKAL LKKTK NSEEF | aa 76-95 | SEQ ID NO: 6 |
| P7: | NSEEF AAAMS RYELK LAIPE | aa 91-110 | SEQ ID NO: 7 |
| P8: | LAIPE GKQVF LYPEK DEPTY | aa 106-125 | SEQ ID NO: 8 |
| P9: | DEPTY ILNIK RGIIS ALLVP | aa 121-140 | SEQ ID NO: 9 |
| P10: | ALLVP PETEE AKQVL FLDTV | aa 136-155 | SEQ ID NO: 10 |
| P11: | FLDTV YGNCS THFTV KTRKG | aa 151-170 | SEQ ID NO: 11 |
| P12: | KTRKG NVATE ISTER DLGQC | aa 166-185 | SEQ ID NO: 12 |
| P13: | DLGQC DRFKP IRTGI SPLAL | aa 181-200 | SEQ ID NO: 13 |
| P14: | SPLAL IKGMT RPLST LISSS | aa 196-215 | SEQ ID NO: 14 |
| P15: | LISSS QSCQY TLDAK RKHVA | aa 211-230 | SEQ ID NO: 15 |
| P16: | RKHVA EAICK EQHLF LPFSY | aa 226-245 | SEQ ID NO: 16 |
| P17: | LPFSY NNKYG MVAQV TQTLK | aa 241-260 | SEQ ID NO: 17 |
| P18: | TQTLK LEDTP KINSR FFGEG | aa 256-275 | SEQ ID NO: 18 |
| P19: | FFGEG TKKMG LAFES TKSTS | aa 271-290 | SEQ ID NO: 19 |
| P20: | TKSTS PPKQA EAVLK TLQEL | aa 286-305 | SEQ ID NO: 20 |
| P21: | TLQEL KKLTI SEQNI QRANL | aa 301-320 | SEQ ID NO: 21 |
| P22: | QRANL FNKLV TELRG LSDEA | aa 316-335 | SEQ ID NO: 22 |
| P23: | LSDEA VTSLL PQLIE VSSPI | aa 331-350 | SEQ ID NO: 23 |
| P24: | VSSPI TLQAL VQCGQ PQCST | aa 346-365 | SEQ ID NO: 24 |
| P25: | PQCST HILQW LKRVH ANPLL | aa 361-380 | SEQ ID NO: 25 |
| P26: | ANPLL IDVVT YLVAL IPEPS | aa 376-395 | SEQ ID NO: 26 |
| P27: | IPEPS AQQLR EIFNM ARDQR | aa 391-410 | SEQ ID NO: 27 |
| P28: | ARDQR SRATL YALSH AVNNY | aa 406-425 | SEQ ID NO: 28 |
| P29: | AVNNY HKTNP TGTQE LLDIA | aa 421-440 | SEQ ID NO: 29 |
| P30: | LLDIA NYLME QIQDD CTGDE | aa 436-455 | SEQ ID NO: 30 |
| P31: | CTGDE DYTYL ILRVI GNMGQ | aa 451-470 | SEQ ID NO: 31 |
| P32: | GNMGQ TMEQL TPELK SSILK | aa 466-485 | SEQ ID NO: 32 |
| P33: | SSILK CVQST KPSLM IQKAA | aa 481-500 | SEQ ID NO: 33 |
| P34: | IQKAA IQALR KMEPK DKDQE | aa 496-515 | SEQ ID NO: 34 |
| P35: | DKDQE VLLQT FLDDA SPGDK | aa 511-530 | SEQ ID NO: 35 |
| P36: | SPGDK RLAAY LMLMR SPSQA | aa 526-545 | SEQ ID NO: 36 |
| P37: | SPSQA DINKI VQILP WEQNE | aa 541-560 | SEQ ID NO: 37 |
| P38: | WEQNE QVKNF VASHI ANILN | aa 556-575 | SEQ ID NO: 38 |
| P39: | ANILN SEELD IQDLK KLVKE | aa 571-590 | SEQ ID NO: 39 |
| P40: | KLVKE ALKES QLPTV MDFRK | aa 586-605 | SEQ ID NO: 40 |
| P41: | MDFRK FSRNY QLYKS VSLPS | aa 601-620 | SEQ ID NO: 41 |
| P42: | VSLPS LDPAS AKIEG NLIFD | aa 616-635 | SEQ ID NO: 42 |
| P43: | NLIFD PNNYL PKESM LKTTL | aa 631-650 | SEQ ID NO: 43 |
| P44: | LKTTL TAFGF ASADL IEIGL | aa 646-665 | SEQ ID NO: 44 |
| P45: | IEIGL EGKGF EPTLE ALFGK | aa 661-680 | SEQ ID NO: 45 |
| P46: | ALFGK QGFFP DSVNK ALYWV | aa 676-695 | SEQ ID NO: 46 |
| P47: | ALYWV NGQVP DGVSK VLVDH | aa 691-710 | SEQ ID NO: 47 |
| P48: | VLVDH FGYTK DDKHE QDMVN | aa 706-725 | SEQ ID NO: 48 |
| P49: | QDMVN GIMLS VEKLI KDLKS | aa 721-740 | SEQ ID NO: 49 |
| P50: | KDLKS KEVPE ARAYL RILGE | aa 736-755 | SEQ ID NO: 50 |
| P51: | RILGE ELGFA SLHDL QLLGK | aa 751-770 | SEQ ID NO: 51 |
| P52: | QLLGK LLLMG ARTLQ GIPQM | aa 766-785 | SEQ ID NO: 52 |
| P53: | GIPQM IGEVI RKGSK NDFFL | aa 781-800 | SEQ ID NO: 53 |
| P54: | NDFFL HYIFM ENAFE LPTGA | aa 796-815 | SEQ ID NO: 54 |
| P55: | LPTGA GLQLQ ISSSG VIAPG | aa 811-830 | SEQ ID NO: 55 |
| P56: | VIAPG AKAGV KLEVA NMQAE | aa 826-845 | SEQ ID NO: 56 |
| P57: | NMQAE LVAKP SVSVE FVTNM | aa 841-860 | SEQ ID NO: 57 |
| P58: | FVTNM GIIIP DFARS GVQMN | aa 856-875 | SEQ ID NO: 58 |
| P59: | GVQMN TNFFH ESGLE AHVAL | aa 871-890 | SEQ ID NO: 59 |
| P60: | AHVAL KAGKL KFIIP SPKRP | aa 886-905 | SEQ ID NO: 60 |
| P61: | SPKRP VKLLS GGNTL HLVST | aa 901-920 | SEQ ID NO: 61 |
| P62: | HLVST TKTEV IPPLI ENRQS | aa 916-935 | SEQ ID NO: 62 |
| P63: | ENRQS WSVCK QVFPG LNYCT | aa 931-950 | SEQ ID NO: 63 |
| P64: | LNYCT SGAYS NASST DSASY | aa 946-965 | SEQ ID NO: 64 |
| P65: | DSASY YPLTG DTRLE LELRP | aa 961-980 | SEQ ID NO: 65 |
| P66: | LELRP TGEIE QYSVS ATYEL | aa 976-995 | SEQ ID NO: 66 |
| P67: | ATYEL QREDR ALVDT LKFVT | aa 991-1010 | SEQ ID NO: 67 |
| P68: | LKFVT QAEGA KQTEA TMTFK | aa 1006-1025 | SEQ ID NO: 68 |
| P69: | TMTFK YNRQS MTLSS EVQIP | aa 1021-1040 | SEQ ID NO: 69 |
| P70: | EVQIP DFDVD LGTIL RVNDE | aa 1036-1055 | SEQ ID NO: 70 |
| P71: | RVNDE STEGK TSYRL TLDIQ | aa 1051-1070 | SEQ ID NO: 71 |
| P72: | TLDIQ NKKIT EVALM GHLSC | aa 1066-1085 | SEQ ID NO: 72 |
| P73: | GHLSC DTKEE RKIKG VISIP | aa 1081-1100 | SEQ ID NO: 73 |
| P74: | VISIP RLQAE ARSEI LAHWS | aa 1096-1115 | SEQ ID NO: 74 |
| P75: | LAHWS PAKLL LQMDS SATAY | aa 1111-1130 | SEQ ID NO: 75 |
| P76: | SATAY GSTVS KRVAW HYDEE | aa 1126-1145 | SEQ ID NO: 76 |
| P77: | HYDEE KIEFE WNTGT NVDTK | aa 1141-1160 | SEQ ID NO: 77 |
| P78: | NVDTK KMTSN FPVDL SDYPK | aa 1156-1175 | SEQ ID NO: 78 |
| P79: | SDYPK SLHMY ANRLL DHRVP | aa 1171-1190 | SEQ ID NO: 79 |
| P80: | DHRVP ETDMT FRHVG SKLIV | aa 1186-1205 | SEQ ID NO: 80 |
| P81: | SKLIV AMSSW LQKAS GSLPY | aa 1201-1220 | SEQ ID NO: 81 |
| P82: | GSLPY TQTLQ DHLNS LKEFN | aa 1216-1235 | SEQ ID NO: 82 |
| P83: | LKEFN LQNMG LPDFH IPENL | aa 1231-1250 | SEQ ID NO: 83 |
| P84: | IPENL FLKSD GRVKY TLNKN | aa 1246-1260 | SEQ ID NO: 84 |
| P85: | TLNKN SLKIE IPLPF GGKSS | aa 1261-1280 | SEQ ID NO: 85 |
| P86: | GGKSS RDLKM LETVR TPALH | aa 1276-1295 | SEQ ID NO: 86 |
| P87: | TPALH FKSVG FHLPS REFQV | aa 1291-1310 | SEQ ID NO: 87 |
| P88: | REFQV PTFTI PKLYQ LQVPL | aa 1306-1325 | SEQ ID NO: 88 |
| P89: | LQVPL LGVLD LSTNV YSNLY | aa 1321-1340 | SEQ ID NO: 89 |
| P90: | YSNLY NWSAS YSGGN TSTDH | aa 1336-1355 | SEQ ID NO: 90 |
| P91: | TSTDH FSLRA RYHMK ADSVV | aa 1351-1370 | SEQ ID NO: 91 |
| P92: | ADSVV DLLSY NVQGS GETTY | aa 1366-1385 | SEQ ID NO: 92 |
| P93: | GETTY DHKNT FTLSC DGSLR | aa 1381-1400 | SEQ ID NO: 93 |
| P94: | DGSLR HKFLD SNIKF SHVEK | aa 1396-1415 | SEQ ID NO: 94 |
| P95: | SHVEK LGNNP VSKGL LIFDA | aa 1411-1430 | SEQ ID NO: 95 |
| P96: | LIFDA SSSWG PQMSA SVHLD | aa 1426-1445 | SEQ ID NO: 96 |
| P97: | SVHLD SKKKQ HLFVK EVKID | aa 1441-1460 | SEQ ID NO: 97 |
| P98: | EVKID GQFRV SSFYA KGTYG | aa 1456-1475 | SEQ ID NO: 98 |
| P99: | KGTYG LSCQR DPNTG RLNGE | aa 1471-1490 | SEQ ID NO: 99 |
| P100: | RLNGE SNLRF NSSYL QGTNQ | aa 1486-1505 | SEQ ID NO: 100 |
| P101: | QGTNQ ITGRY EDGTL SLTST | aa 1501-1520 | SEQ ID NO: 101 |
| P102: | SLTST SDLQS GIIKN TASLK | aa 1516-1535 | SEQ ID NO: 102 |
| P103: | TASLK YENYE LTLKS DTNGK | aa 1531-1550 | SEQ ID NO: 103 |
| P104: | DTNGK YKNFA TSNKM DMTFS | aa 1546-1565 | SEQ ID NO: 104 |
| P105: | DMTFS KQNAL LRSEY QADYE | aa 1561-1580 | SEQ ID NO: 105 |
| P106: | QADYE SLRFF SLLSG SLNSH | aa 1576-1595 | SEQ ID NO: 106 |
| P107: | SLNSH GLELN ADILG TDKIN | aa 1591-1610 | SEQ ID NO: 107 |
| P108: | TDKIN SGAHK ATLRI GQDGI | aa 1606-1625 | SEQ ID NO: 108 |
| P109: | GQDGI STSAT TNLKC SLLVL | aa 1621-1640 | SEQ ID NO: 109 |
| P110: | SLLVL ENELN AELGL SGASM | aa 1636-1655 | SEQ ID NO: 110 |
| P111: | SGASM KLTTN GRFRE HNAKF | aa 1651-1670 | SEQ ID NO: 111 |
| P112: | HNAKF SLDGK AALTE LSLGS | aa 1666-1685 | SEQ ID NO: 112 |
| P113: | LSLGS AYQAM ILGVD SKNIF | aa 1681-1700 | SEQ ID NO: 113 |
| P114: | SKNIF NFKVS QEGLK LSNDM | aa 1696-1715 | SEQ ID NO: 114 |
| P115: | LSNDM MGSYA EMKFD HTNSL | aa 1711-1730 | SEQ ID NO: 115 |
| P116: | HTNSL NIAGL SLDFS SKLDN | aa 1726-1745 | SEQ ID NO: 116 |
| P117: | SKLDN IYSSD KFYKQ TVNLQ | aa 1741-1760 | SEQ ID NO: 117 |
| P118: | TVNLQ LQPYS LVTTL NSDLK | aa 1756-1775 | SEQ ID NO: 118 |
| P119: | NSDLK YNALD LTNNG KLRLE | aa 1771-1790 | SEQ ID NO: 119 |
| P120: | KLRLE PLKLH VAGNL KGAYQ | aa 1786-1805 | SEQ ID NO: 120 |
| P121: | KGAYQ NNEIK HIYAI SSAAL | aa 1801-1820 | SEQ ID NO: 121 |
| P122: | SSAAL SASYK ADTVA KVQGV | aa 1816-1835 | SEQ ID NO: 122 |
| P123: | KVQGV EFSHR LNTDI AGLAS | aa 1831-1850 | SEQ ID NO: 123 |
| P124: | AGLAS AIDMS TNYNS DSLHF | aa 1846-1865 | SEQ ID NO: 124 |
| P125: | DSLHF SNVFR SVMAP FTMTI | aa 1861-1880 | SEQ ID NO: 125 |
| P126: | FTMTI DAHTN GNGKL ALWGE | aa 1876-1895 | SEQ ID NO: 126 |
| P127: | ALWGE HTGQL YSKFL LKAEP | aa 1891-1910 | SEQ ID NO: 127 |
| P128: | LKAEP LAFTF SHDYK GSTSH | aa 1906-1925 | SEQ ID NO: 128 |
| P129: | GSTSH HLVSR KSISA ALEHK | aa 1921-1940 | SEQ ID NO: 129 |
| P130: | ALEHK VSALL TPAEQ TGTWK | aa 1936-1955 | SEQ ID NO: 130 |
| P131: | TGTWK LKTQF NNNEY SQDLD | aa 1951-1970 | SEQ ID NO: 131 |
| P132: | SQDLD AYNTK DKIGV ELTGR | aa 1966-1985 | SEQ ID NO: 132 |
| P133: | ELTGR TLADL TLLDS PIKVP | aa 1981-2000 | SEQ ID NO: 133 |
| P134: | PIKVP LLLSE PINII DALEM | aa 1996-2015 | SEQ ID NO: 134 |
| P135: | DALEM RDAVE KPQEF TIVAF | aa 2011-2030 | SEQ ID NO: 135 |
| P136: | TIVAF VKYDK NQDVH SINLP | aa 2026-2045 | SEQ ID NO: 136 |
| P137: | SINLP FFLTL QEYFE RNRQT | aa 2041-2060 | SEQ ID NO: 137 |
| P138: | RNRQT IIVVV ENVQR NLKHI | aa 2056-2075 | SEQ ID NO: 138 |
| P139: | NLKHI NIDQF VRKYR AALGK | aa 2071-2090 | SEQ ID NO: 139 |
| P140: | AALGK LPQQA NDYLN SFNWE | aa 2086-2105 | SEQ ID NO: 140 |
| P141: | SFNWE RQVSH AKEKL TALTK | aa 2101-2120 | SEQ ID NO: 141 |
| P142: | TALTK KYRIT ENDIQ IALDD | aa 2116-2135 | SEQ ID NO: 142 |
| P143: | IALDD AKINF NEKLS QLQTY | aa 2131-2150 | SEQ ID NO: 143 |
| P144: | QLQTY MIQFD QYIKD SYDLH | aa 2146-2165 | SEQ ID NO: 144 |
| P145: | SYDLH DLKIA IANII DEIIE | aa 2161-2180 | SEQ ID NO: 145 |
| P146: | DEIIE KLKSL DEHYH IRVNL | aa 2176-2195 | SEQ ID NO: 146 |
| P147: | IRVNL VKTIH DLHLF IENID | aa 2191-2210 | SEQ ID NO: 147 |
| P148: | IENID FNKSG SSTAS WIQNV | aa 2206-2225 | SEQ ID NO: 148 |
| P149: | WIQNV DTKYQ IRIQI QEKLQ | aa 2221-2240 | SEQ ID NO: 149 |
| P150: | QEKLQ QLKRH IQNID IQHLA | aa 2236-2255 | SEQ ID NO: 150 |
| P151: | IQHLA GKLKQ HIEAI DVRVL | aa 2251-2270 | SEQ ID NO: 151 |
| P152: | DVRVL LDQLG TTISF ERIND | aa 2266-2285 | SEQ ID NO: 152 |
| P153: | ERIND VLEHV KHFVI NLIGD | aa 2281-2300 | SEQ ID NO: 153 |
| P154: | NLIGD FEVAE KINAF RAKVH | aa 2296-2315 | SEQ ID NO: 154 |
| P155: | RAKVH ELIER YEVDQ QIQVL | aa 2311-2330 | SEQ ID NO: 155 |
| P156: | QIQVL MDKLV ELTHQ YKLKE | aa 2326-2345 | SEQ ID NO: 156 |
| P157: | YKLKE TIQKL SNVLQ QVKIK | aa 2341-2360 | SEQ ID NO: 157 |
| P158: | QVKIK DYFEK LVGFI DDAVK | aa 2356-2375 | SEQ ID NO: 158 |
| P159: | DDAVK KLNEL SFKTF IEDVN | aa 2371-2390 | SEQ ID NO: 159 |
| P160: | IEDVN KFLDM LIKKL KSFDY | aa 2386-2405 | SEQ ID NO: 160 |
| P161: | KSFDY HQFVD ETNDK IREVT | aa 2401-2420 | SEQ ID NO: 161 |
| P162: | IREVT QRLNG EIQAL ELPQK | aa 2416-2435 | SEQ ID NO: 162 |
| P163: | ELPQK AEALK LFLEE TKATV | aa 2431-2450 | SEQ ID NO: 163 |
| P164: | TKATV AVYLE SLQDT KITLI | aa 2446-2465 | SEQ ID NO: 164 |
| P165: | KITLI INWLQ EALSS ASLAH | aa 2461-2480 | SEQ ID NO: 165 |
| P166: | ASLAH MKAKF RETLE DTRDR | aa 2476-2495 | SEQ ID NO: 166 |
| P167: | DTRDR MYQMD IQQEL QRYLS | aa 2491-2510 | SEQ ID NO: 167 |
| P168: | QRYLS LVGQV YSTLV TYISD | aa 2506-2515 | SEQ ID NO: 168 |
| P169: | TYISD WWTLA AKNLT DFAEQ | aa 2521-2540 | SEQ ID NO: 169 |
| P170: | DFAEQ YSIQD WAKRM KALVE | aa 2536-2555 | SEQ ID NO: 170 |
| P171: | KALVE QGFTV PEIKT ILGTM | aa 2551-2570 | SEQ ID NO: 171 |
| P172: | ILGTM PAFEV SLQAL QKATF | aa 2566-2585 | SEQ ID NO: 172 |
| P173: | QKATF QTPDF IVPLT DLRIP | aa 2581-2600 | SEQ ID NO: 173 |
| P174: | DLRIP SVQIN FKDLK NIKIP | aa 2596-2615 | SEQ ID NO: 174 |
| P175: | NIKIP SRFST PEFTI LNTFH | aa 2611-2630 | SEQ ID NO: 175 |
| P176: | LNTFH IPSFT IDFVE MKVKI | aa 2626-2645 | SEQ ID NO: 176 |
| P177: | MKVKI IRTID QMQNS ELQWP | aa 2641-2660 | SEQ ID NO: 177 |
| P178: | ELQWP VPDIY LRDLK VEDIP | aa 2656-2675 | SEQ ID NO: 178 |
| P179: | VEDIP LARIT LPDFR LPEIA | aa 2671-2690 | SEQ ID NO: 179 |
| P180: | LPEIA IPEFI IPTLN LNDFQ | aa 2686-2705 | SEQ ID NO: 180 |
| P181: | LNDFQ VPDLH IPEFQ LPHIS | aa 2701-2720 | SEQ ID NO: 181 |
| P182: | LPHIS HTIEV PTFGK LYSIL | aa 2716-2735 | SEQ ID NO: 182 |
| P183: | LYSIL KIQSP LFTLD ANADI | aa 2731-2750 | SEQ ID NO: 183 |
| P184: | ANADI GNGTT SANEA GIAAS | aa 2746-2765 | SEQ ID NO: 184 |
| P185: | GIAAS ITAKG ESKLE VLNFD | aa 2761-2780 | SEQ ID NO: 185 |
| P186: | VLNFD FQANA QLSNP KINPL | aa 2776-2795 | SEQ ID NO: 186 |
| P187: | KINPL ALKES VKFSS KYLRT | aa 2791-2810 | SEQ ID NO: 187 |
| P188: | KYLRT EHGSE MLFFG NAIEG | aa 2806-2825 | SEQ ID NO: 188 |
| P189: | NAIEG KSNTV ASLHT EKNTL | aa 2821-2840 | SEQ ID NO: 189 |
| P190: | EKNTL ELSNG VIVKI NNQLT | aa 2836-2855 | SEQ ID NO: 190 |
| P191: | NNQLT LDSNT KYFHK LNIPK | aa 2851-2870 | SEQ ID NO: 191 |
| P192: | LNIPK LDFSS QADLR NEIKT | aa 2866-2885 | SEQ ID NO: 192 |
| P193: | NEIKT LLKAG HIAWT SSGKG | aa 2881-2900 | SEQ ID NO: 193 |
| P194: | SSGKG SWKWA CPRFS DEGTH | aa 2896-2915 | SEQ ID NO: 194 |
| P195: | DEGTH ESQIS FTIEG PLTSF | aa 2911-2930 | SEQ ID NO: 195 |
| P196: | PLTSF GLSNK INSKH LRVNQ | aa 2926-2945 | SEQ ID NO: 196 |
| P197: | LRVNQ NLVYE SGSLN FSKLE | aa 2941-2960 | SEQ ID NO: 197 |
| P198: | FSKLE IQSQV DSQHV GHSVL | aa 2956-2975 | SEQ ID NO: 198 |
| P199: | GHSVL TAKGM ALFGE GKAEF | aa 2971-2990 | SEQ ID NO: 199 |
| P200: | GKAEF TGRHD AHLNG KVIGT | aa 2986-3005 | SEQ ID NO: 200 |
| P201: | KVIGT LKNSL FFSAQ PFEIT | aa 3001-3020 | SEQ ID NO: 201 |
| P202: | PFEIT ASTNN EGNLK VRFPL | aa 3016-3035 | SEQ ID NO: 202 |
| P203: | VRFPL RLTGK IDFLN NYALF | aa 3031-3050 | SEQ ID NO: 203 |
| P204: | NYALF LSPSA QQASW QVSAR | aa 3046-3065 | SEQ ID NO: 204 |
| P205: | QVSAR FNQYK YNQNF SAGNN | aa 3061-3080 | SEQ ID NO: 205 |
| P206: | SAGNN ENIME AHVGI NGEAN | aa 3076-3095 | SEQ ID NO: 206 |
| P207: | NGEAN LDFLN IPLTI PEMRL | aa 3091-3110 | SEQ ID NO: 207 |
| P208: | PEMRL PYTII TTPPL KDFSL | aa 3106-3125 | SEQ ID NO: 208 |
| P209: | KDFSL WEKTG LKEFL KTTKQ | aa 3121-3140 | SEQ ID NO: 209 |
| P210: | KTTKQ SFDLS VKAQY KKNKH | aa 3136-3155 | SEQ ID NO: 210 |
| P211: | KKNKH RHSIT NPLAV LCEFI | aa 3151-3170 | SEQ ID NO: 211 |
| P212: | LCEFI SQSIK SFDRH FLKNR | aa 3166-3185 | SEQ ID NO: 212 |
| P213: | FEKNR NNALD FVTKS YNETK | aa 3181-3200 | SEQ ID NO: 213 |
| P214: | YNETK IKFDK YKAEK SHDEL | aa 3196-3215 | SEQ ID NO: 214 |
| P215: | SHDEL PRTFQ IPGYT VPVVN | aa 3211-3230 | SEQ ID NO: 215 |
| P216: | VPVVN VEVSP FTIEM SAFGY | aa 3226-3245 | SEQ ID NO: 216 |
| P217: | SAFGY VFPKA VSMPS FSILG | aa 3241-3260 | SEQ ID NO: 217 |
| P218: | FSILG SDVRV PSYTL ILPSL | aa 3256-3275 | SEQ ID NO: 218 |
| P219: | ILPSL ELPVL HVPRN LKLSL | aa 3271-3290 | SEQ ID NO: 219 |
| P220: | LKLSL PHFKE LCTIS HIFIP | aa 3286-3305 | SEQ ID NO: 220 |
| P221: | HIFIP AMGNI TYDFS FKSSV | aa 3301-3320 | SEQ ID NO: 221 |
| P222: | FKSSV ITLNT NAELF NQSDI | aa 3316-3335 | SEQ ID NO: 222 |
| P223: | NQSDI VAHLL SSSSS VIDAL | aa 3331-3350 | SEQ ID NO: 223 |
| P224: | VIDAL QYKLE GTTRL TRKRG | aa 3346-3365 | SEQ ID NO: 224 |
| P225: | TRKRG LKLAT ALSLS NKFVE | aa 3361-3380 | SEQ ID NO: 225 |
| P226: | NKFVE GSHNS TVSLT TKNME | aa 3376-3395 | SEQ ID NO: 226 |
| P227: | TKNME VSVAK TTKAE IPILR | aa 3391-3410 | SEQ ID NO: 227 |
| P228: | IPILR MNFKQ ELNGN TKSKP | aa 3406-3425 | SEQ ID NO: 228 |
| P229: | TKSKP TVSSS MEFKY DFNSS | aa 3421-3440 | SEQ ID NO: 229 |
| P230: | DFNSS MLYST AKGAV DHKLS | aa 3436-3455 | SEQ ID NO: 230 |
| P231: | DHKLS LESLT SYFSI ESSTK | aa 3451-3470 | SEQ ID NO: 231 |
| P232: | ESSTK GDVKG SVLSR EYSGT | aa 3466-3485 | SEQ ID NO: 232 |
| P233: | EYSGT IASEA NTYLN SKSTR | aa 3481-3500 | SEQ ID NO: 233 |
| P234: | SKSTR SSVKL QGTSK IDDIW | aa 3496-3515 | SEQ ID NO: 234 |
| P235: | IDDIW NLEVK ENFAG EATLQ | aa 3511-3530 | SEQ ID NO: 235 |
| P236: | EATLQ RIYSL WEHST KNHLQ | aa 3526-3545 | SEQ ID NO: 236 |
| P237: | KNHLQ LEGLF FTNGE HTSKA | aa 3541-3560 | SEQ ID NO: 237 |
| P238: | HTSKA TLELS PWQMS ALVQV | aa 3556-3575 | SEQ ID NO: 238 |
| P239: | ALVQV HASQP SSFHD FPDLG | aa 3571-3590 | SEQ ID NO: 239 |
| P240: | FPDLG QEVAL NANTK NQKIR | aa 3586-3605 | SEQ ID NO: 240 |
| P241: | NQKIR WKNEV RIHSG SFQSQ | aa 3601-3620 | SEQ ID NO: 241 |
| P242: | SFQSQ VELSN DQEKA HLDIA | aa 3616-3635 | SEQ ID NO: 242 |
| P243: | HLDIA GSLEG HLRFL KNIIL | aa 3631-3650 | SEQ ID NO: 243 |
| P244: | KNIIL PVYDK SLWDF LKLDV | aa 3646-3665 | SEQ ID NO: 244 |
| P245: | LKLDV TTSIG RRQHL RVSTA | aa 3661-3680 | SEQ ID NO: 245 |
| P246: | RVSTA FVYTK NPNGY SFSIP | aa 3676-3695 | SEQ ID NO: 246 |
| P247: | SFSIP VKVLA DKFIT PGLKL | aa 3691-3710 | SEQ ID NO: 247 |
| P248: | PGLKL NDLNS VLVMP TFHVP | aa 3706-3725 | SEQ ID NO: 248 |
| P249: | TFHVP FTDLQ VPSCK LDFRE | aa 3721-3740 | SEQ ID NO: 249 |
| P250: | LDFRE IQIYK KLRTS SFALN | aa 3736-3755 | SEQ ID NO: 250 |
| P251: | SFALN LPTLP EVKFP EVDVL | aa 3751-3770 | SEQ ID NO: 251 |
| P252: | EVDVL TKYSQ PEDSL IPFFE | aa 3766-3785 | SEQ ID NO: 252 |
| P253: | IPFFE ITVPE SQLTV SQFTL | aa 3781-3800 | SEQ ID NO: 253 |
| P254: | SQFTL PKSVS DGIAA LDLNA | aa 3796-3815 | SEQ ID NO: 254 |
| P255: | LDLNA VANKI ADFEL PTIIV | aa 3811-3830 | SEQ ID NO: 255 |
| P256: | PTIIV PEQTI EIPSI KFSVP | aa 3826-3845 | SEQ ID NO: 256 |
| P257: | KFSVP AGIVI PSFQA LTARF | aa 3841-3860 | SEQ ID NO: 257 |
| P258: | LTARF EVDSP VYNAT WSASL | aa 3856-3875 | SEQ ID NO: 258 |
| P259: | WSASL KNKAD YVETV LDSTC | aa 3871-3890 | SEQ ID NO: 259 |
| P260: | LDSTC SSTVQ FLEYE LNVLG | aa 3886-3905 | SEQ ID NO: 260 |
| P261: | LNVLG THKIE DGTLA SKTKG | aa 3901-3920 | SEQ ID NO: 261 |
| P262: | SKTKG TLAHR DFSAE YEEDG | aa 3916-3935 | SEQ ID NO: 262 |
| P263: | YEEDG KFEGL QEWEG KAHLN | aa 3931-3950 | SEQ ID NO: 263 |
| P264: | KAHLN IKSPA FTDLH LRYQK | aa 3946-3965 | SEQ ID NO: 264 |
| P265: | LRYQK DKKGI STSAA SPAVG | aa 3961-3980 | SEQ ID NO: 265 |
| P266: | SPAVG TVGMD MDEDD DFSKW | aa 3976-3995 | SEQ ID NO: 266 |
| P267: | DFSKW NFYYS PQSSP DKKLT | aa 3991-4010 | SEQ ID NO: 267 |
| P268: | DKKLT IFKTE LRVRE SDEET | aa 4006-4025 | SEQ ID NO: 268 |
| P269: | SDEET QIKVN WEEEA ASGLL | aa 4021-4040 | SEQ ID NO: 269 |
| P270: | ASGLL TSLKD NVPKA TGVLY | aa 4036-4055 | SEQ ID NO: 270 |
| P271: | TGVLY DYVNK YHWEH TGLTL | aa 4051-4070 | SEQ ID NO: 271 |
| P272: | TGLTL REVSS KLRRN LQNNA | aa 4066-4085 | SEQ ID NO: 272 |
| P273: | LQNNA EWVYQ GAIRQ IDDID | aa 4081-4100 | SEQ ID NO: 273 |
| P274: | IDDID VRFQK AASGT TGTYQ | aa 4096-4115 | SEQ ID NO: 274 |
| P275: | TGTYQ EWKDK AQNLY QELLT | aa 4111-4130 | SEQ ID NO: 275 |
| P276: | QELLT QEGQA SFQGL KDNVF | aa 4126-4145 | SEQ ID NO: 276 |
| P277: | KDNVF DGLVR VTQKF HMKVK | aa 4141-4160 | SEQ ID NO: 277 |
| P278: | HMKVK HLIDS LIDFL NFPRF | aa 4156-4175 | SEQ ID NO: 278 |
| P279: | NFPRF QFPGK PGIYT REELC | aa 4171-4190 | SEQ ID NO: 279 |
| P280: | REELC TMFIR EVGTV LSQVY | aa 4186-4205 | SEQ ID NO: 280 |
| P281: | LSQVY SKVHN GSEIL FSYFQ | aa 4201-4220 | SEQ ID NO: 281 |
| P282: | FSYFQ DLVIT LPFEL RKHKL | aa 4216-4235 | SEQ ID NO: 282 |
| P283: | RKHKL IDVIS MYREL LKDLS | aa 4231-4250 | SEQ ID NO: 283 |
| P284: | LKDLS KEAQE VFKAI QSLKT | aa 4246-4265 | SEQ ID NO: 284 |
| P285: | QSLKT TEVLR NLQDL LQFIF | aa 4261-4280 | SEQ ID NO: 285 |
| P286: | LQFIF QLIED NIKQL KEMKF | aa 4276-4295 | SEQ ID NO: 286 |
| P287: | KEMKF TYLIN YIQDE INTIF | aa 4291-4310 | SEQ ID NO: 287 |
| P288: | INTIF NDYIP YVFKL LKENL | aa 4306-4325 | SEQ ID NO: 288 |
| P289: | LKENL CLNLH KFNEF IQNEL | aa 4321-4340 | SEQ ID NO: 289 |
| P290: | IQNEL QEASQ ELQQI HQYIM | aa 4336-4355 | SEQ ID NO: 290 |
| P291: | HQYIM ALREE YFDPS IVGWT | aa 4351-4370 | SEQ ID NO: 291 |
| P292: | IVGWT VKYYE LEEKI VSLIK | aa 4366-4385 | SEQ ID NO: 292 |
| P293: | VSLIK NLLVA LKDFH SEYIV | aa 4381-4400 | SEQ ID NO: 293 |
| P294: | SEYIV SASNF TSQLS SQVEQ | aa 4396-4415 | SEQ ID NO: 294 |
| P295: | SQVEQ FLHRN IQEYL SILTD | aa 4411-4430 | SEQ ID NO: 295 |
| P296: | SILTD PDGKG KEKIA ELSAT | aa 4426-4445 | SEQ ID NO: 296 |
| P297: | ELSAT AQEII KSQAI ATKKI | aa 4441-4460 | SEQ ID NO: 297 |
| P298: | TKKII SDYHQ QFRYK LQDFS | aa 4457-4476 | SEQ ID NO: 298 |
| P299: | LQDFS DQLSD YYEKF IAESK | aa 4472-4491 | SEQ ID NO: 299 |
| P300: | IAESK RLIDL SIQNY HTFLI | aa 4487-4506 | SEQ ID NO: 300 |
| P301: | HTFLI YITEL LKKLQ STTVM | aa 4502-4521 | SEQ ID NO: 301 |
| P302: | STTVM NPYMK LAPGE LTIIL | aa 4517-4536 | SEQ ID NO: 302 |

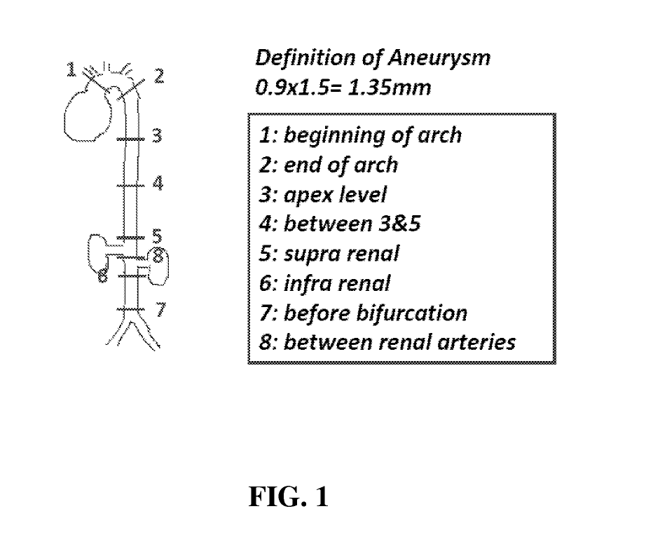

The full length sequence of ApoB100 can be found in various publications such as San-Hwan Chen et al The complete cDNA and amino acid sequence of Human Apolipoprotein B100 Journal of Biological Chemistry 1986 Vol. 261No 28, Issue of October 5, 12918-12921 (see in particular FIG. 1) herein incorporated by reference in its entirety.

### Example 2

### Immunization with an apoB-100 Immunogenic Fragments Reduced Aortic Aneurysm Rupture

Male apoE KO mice were subcutaneously immunized at 7, 10, and 12 weeks of age with either Group 1: P210/cBSA conjugate using alum as adjuvant (100 μg P210); Group 2: control-100 μg of cBSA/alum (cBSA); Group 3: control PBS (PBS). Fourteen P210, 17 cBSA, 16 PBS, and 8 Saline injected mice were examined.

AngII (1000 ng/Kg/min) was delivered by a subcutaneous osmotic pump implanted at 10 weeks of age for 4 weeks to cause aneurysms in all three groups. Saline was delivered to the control group. Mice were sacrificed at 14 weeks of age of age. The mice were fed normal chow for the duration of experiment.

Aneurysm formation (including rupture) and incidence were investigated. The results are illustrated in Table 3A and Table 3B below.

TABLE 3A

| | | | | | | Rupture |
|---|---|---|---|---|---|---|
| | | | | | Aneurysm | incidence |
| Pump | Group | Total | alive | Rupture | (include Rupture) | (%, total) |
| AngII | p210 | 14 | 13 | 1 | 8 | 7.1 |
| | cBSA | 17 | 14 | 3 | 14 | 17.7 |
| | PBS | 16 | 11 | 5 | 11 | 31.3 |
| Saline | Saline | 8 | 8 | 0 | 0 | 0 |

TABLE 3B

| | | Number of mice in | Aneurysm | Survival at |
|---|---|---|---|---|
| Pump | Group | each group | incidence | 28 days |
| Ang II | P210 | 42 | 54.8% | 90.5% |
| | cBSA | 46 | 84.8% | 69.6% |
| | PBS | 37 | 81.1% | 64.9% |
| Saline | Saline | 8 | 0% | 100% |

As illustrated in the above Tables, P210 immunization reduced rupture incidence. Immunization with apoB-100 related peptide P210 reduced rupture incidence 7.1% from 17.7% with cBSA as a control and 31.3% using PBS as a control.

A possible mechanism of action provided herein for guidance purposes only and not intended to be limiting is that p210 immunization reduces BP; 2. Effect of p210 immunization is mediated by CD8 to a same or comparable extent detected for reduction of atherosclerosis illustrated in the following examples. Accordingly, ability to elicit a T cell response is specific for p210 (antigen specificity) and other apoB-100 peptides are expected to show similar antigen-specific CD8 effect.

### Example 3

### Immunization with an Immunogenic Fragment of ApoB-100 Reduces Aortic Aneurysmal Segment Formation

Male apoE KO mice were subcutaneously immunized at 7, 10, and 12 weeks of age with either Group 1: P210/cBSA conjugate using alum as adjuvant (100 μg P210); Group 2: control-100 μg of cBSA/alum (cBSA); Group 3: control PBS (PBS). 42 P210, 46 cBSA, 37 PBS, and 8 Saline injected mice were examined.

AngII (1000 ng/Kg/min) was delivered by a subcutaneous osmotic pump implanted at 10 weeks of age for 4 weeks to cause aneurysms in all three groups. Saline was delivered to the control group. Mice were sacrificed at 14 weeks of age of age. The male apoE KO mice were fed normal chow for the duration of experiment.

The measurement of the aorta was taken at 8 segments: 1) beginning of arch, 2) end of arch, 3) apex level, 4) between 3 & 5, 5) supra renal, 6) infra renal, 7) before bifurcation, and 8) between renal arteries (see schematic illustration of FIG. 1).

The average diameters of each segment illustrated in FIG. 1 are reported in Table 4 below, wherein the segmental aneurysms are circled.

TABLE 4

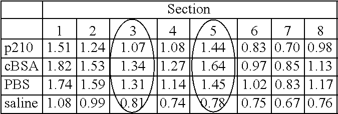

A further elaboration of the data of Table 4, illustrated in Table 5 below suggests that P210 immunization significantly reduces aneurysmal section formation. Whereas the aneurysmal segment/total segment percentage is 29.6% for cBSA controls and 23.4% for PBS controls, P210 immunization reduced the aneurysmal segment/total segment percentage to 14.3%.

TABLE 5

| | Aneurysmal segment/total segment |
|---|---|
| | (%) |
| p210 | 14.3 |
| cBSA | 29.6 |
| PBS | 23.4 |

### Example 4

### Immunization with an Immunogenic Fragment of ApoB100 Reduces Mortality Associated with Aortic Aneurysmal Rupture

Male apoE KO mice were subcutaneously immunized at 7, 10, and 12 weeks of age with either Group 1: P210/cBSA conjugate using alum as adjuvant (100 μg P210); Group 2: control-100 μg of cBSA/alum (cBSA); Group 3: control PBS (PBS). 42 P210, 46 cBSA, 37 PBS, and 8 Saline injected mice were examined.

AngII (1000 ng/Kg/min) was delivered by a subcutaneous osmotic pump implanted at 10 weeks of age for 4 weeks to cause aneurysms in all three groups. Saline was delivered to the control group. Mice were sacrificed at 14 weeks of age of age. 42 P210, 46 cBSA, 37 PBS, and 8 Saline injected mice were examined. Mice were sacrificed at 14 weeks of age. The male apoE KO mice were fed normal chow for the duration of experiment.

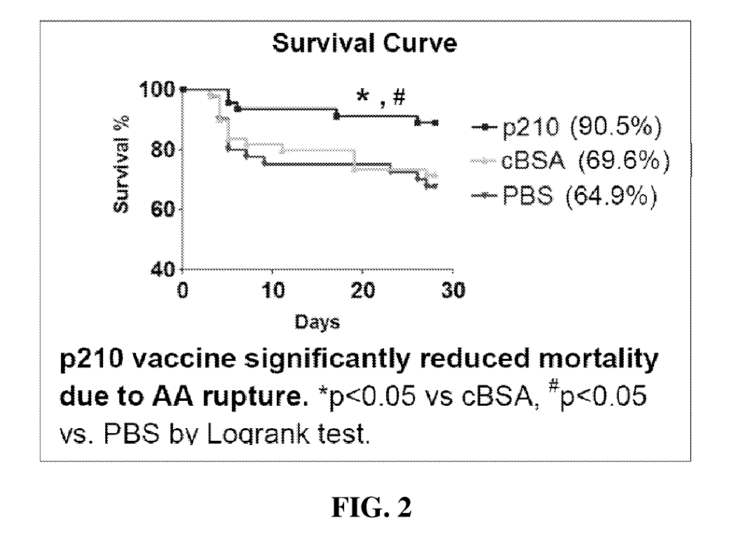

The results are illustrated in the chart of FIG. 2, which shows survival of the mice treated with p210 over the control groups. The survival rate was 90.5% for P210, 69.6% for cBSA, 64.9% for PBS, and 0% for the saline control at 28 days after implantation of an osmotic pump for angiotensin II infusion to elicit aneurysm formation, as shown in the illustration of FIG. 2.

### Example 5

### Athero-Protective Effects of p210 Immunization

The vaccine preparation consisted of the p210 peptide (Euro-Diagnostica AB, Sweden) conjugated to cationic bovine serum albumin (cBSA) as carrier using a method described previously^{3;4}. Alum was used as adjuvant and mixed with peptide/cBSA conjugated with 1:1 ratio in volume. Peptide conjugation was performed on the day of immunization and freshly mixed with alum just prior to each immunization. Mice fed normal chow diet received subcutaneous primary immunization in the dorsal area between scapulas at 6-7 weeks of age, followed by a booster at 10 and 12 weeks of age. One week after the last booster, diet was switched to high cholesterol chow (TD 88137, Harlan-Teklad) and continued until euthanasia at the age of 25 weeks.

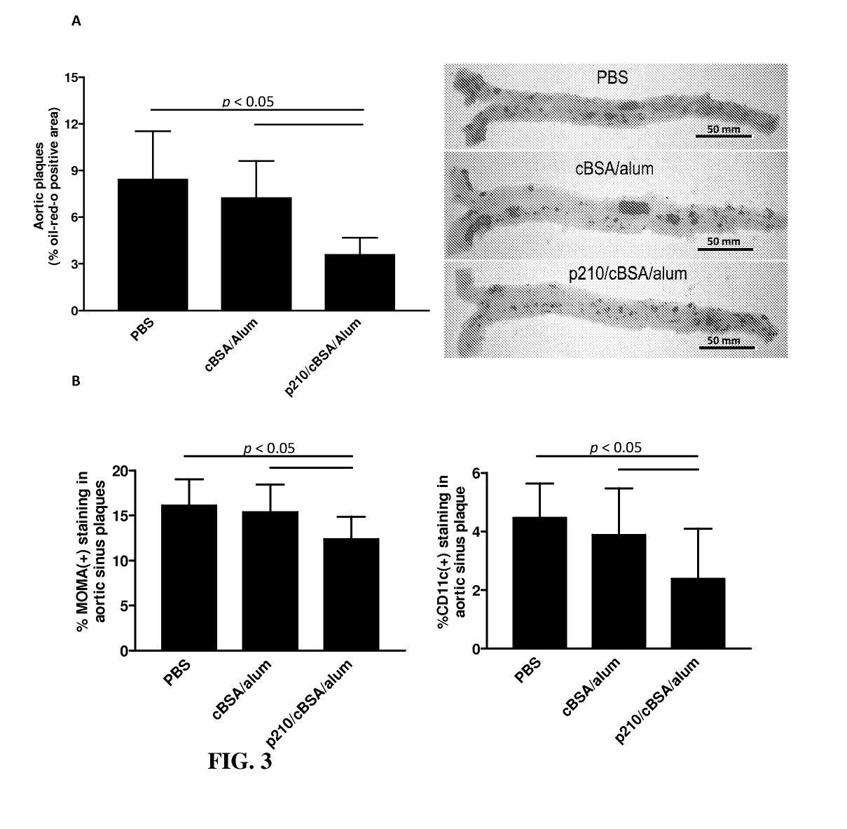

Immunization with p210 reduced aortic atherosclerosis by 57% and 50% compared to PBS and cBSA/Alum group, respectively (FIG. 3A) without affecting circulating cholesterol levels or body weight (Table 6).

TABLE 6
Circulating level of cholesterol and body weight of mice
from PBS, cBSA/alum and p210/cBSA/alum group

| | PBS | cBSA/alum | P210/cBSA/alum | P value |
|---|---|---|---|---|
| | (n = 10) | (n = 10) | (n = 10) | (ANOVA) |
| Cholesterol | 1503 ± 485 | 1395 ± 420 | 1135 ± 382 | 0.17 |
| (mg/dl) | | | | |
| Body weight | 37.9 ± 5.4 | 34.8 ± 5.4 | 34.3 ± 6.5 | 0.33 |
| (gm) | | | | |

The aortic sinus plaques from p210/cBSA/alum group contained significantly reduced macrophage and DC immuno-reactivity assessed by MOMA-2 and CD11c immuno-staining, respectively (FIG. 3B) with no difference in the atherosclerotic lesions (PBS group 0.40±0.13 mm², n=10; cBSA/alum group 0.42±0.09 mm², n=10; p210/cBSA/alum group 0.40±0.08 mm², n=9).

### Example 6

### Characterization of p210-Immunization Elicited Immune Responses

Since DCs are the major cell type upstream to both cellular and humoral immune responses, Applicants determined if these cells were affected by the immunization strategy. Cells from the subcutaneous immunization sites were isolated for flow cytometric analysis one week after primary immunization. The PBS group could not be included in this analysis because mice receiving PBS injection did not develop swelling or cell accumulation at the injection site.

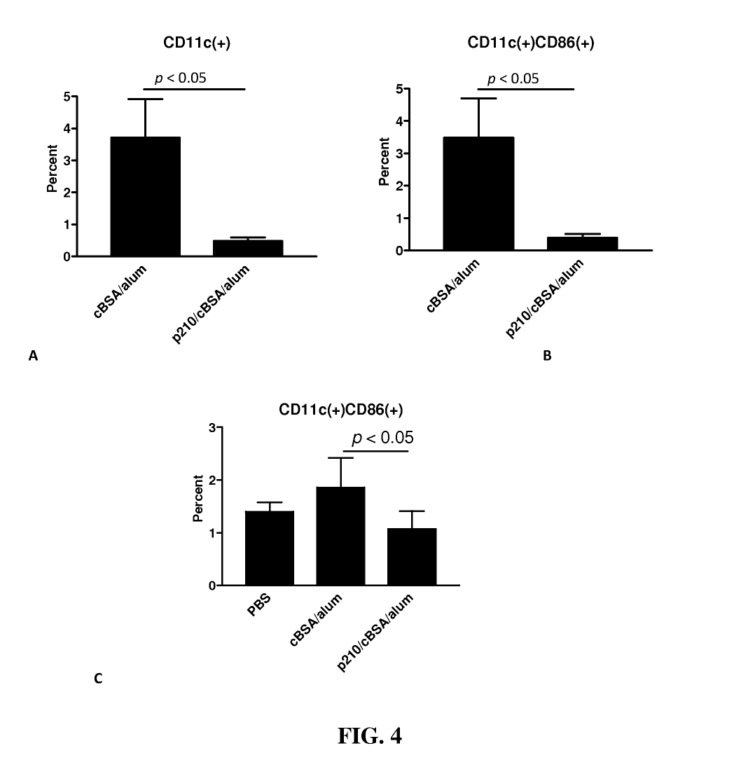

There were significantly fewer CD11c(+) and CD11c(+)CD86(+) cells in p210/cBSA/alum group compared to cBSA/alum group at the immunization site (FIGS. 4A and 4B). When flow cytometry was performed on LN cells 1 week after the third immunization, CD11c(+)CD86(+) cells were also significantly reduced compared with cBSA/alum group (FIG. 4C).

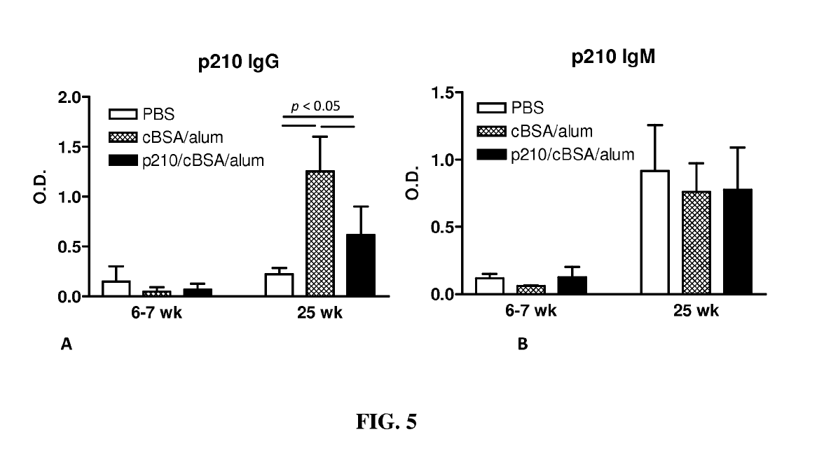

Applicants next assessed antibody response to define the humoral immune response against p210. Before immunization all 3 groups of mice had low levels of IgG titers against p210. At euthanasia, the IgG titer against p210 remained low in the PBS group but was significantly increased in cBSA/alum group. Immunization with p210/cBSA/alum resulted in increased p210 IgG titer compared with PBS group but was significantly reduced compared with cBSA/alum group (FIG. 5A). In contrast to p210 IgG response, there was a significant increase in p210 IgM titer in all groups (FIG. 5B), suggesting an endogenous immune response against p210.

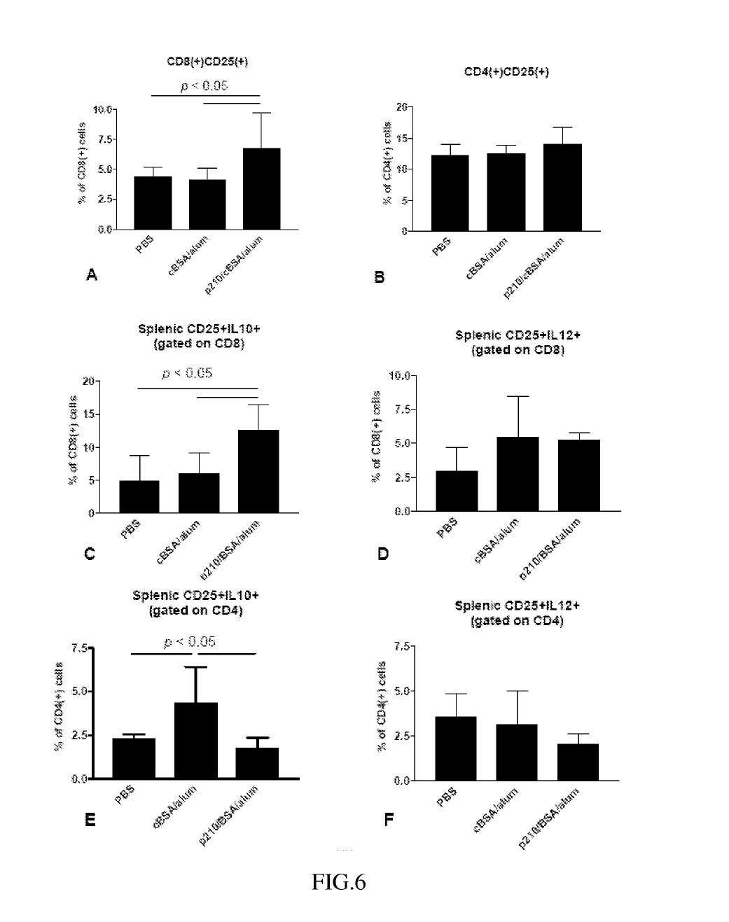

The IL-2Rcc (CD25) is a well-defined lymphocyte activation marker. Applicants therefore analyzed the expression of CD25 on CD4(+) or CD8(+) T-cells from superficial cervical and axillary lymph nodes (LN) from mice one week after primary immunization to assess the T-cell immune response. CD8(+)CD25(+) T-cell population in the lymph nodes was significantly higher in p210/cBSA/alum group when compared to that of PBS or cBSA/alum groups (FIG. 6A) whereas CD4(+)CD25(+) T-cells in the lymph nodes (FIG. 6B) did not differ among 3 groups.

There was a significantly larger population of splenic CD8(+)CD25(+)IL-10(+) T-cells in p210/cBSA/alum group when compared to PBS or cBS A/alum groups (FIG. 6C) without difference in splenic CD8(+)CD25(+)IL12(+) T-cells among 3 groups (FIG. 6D). Splenic CD4(+)CD25(+)IL-10(+) T-cell population significantly increased in the cBSA/alum group. However, this increased response was significantly attenuated by the p210/cBSA/alum immunization (FIG. 6E); whereas splenic CD4(+)CD25(+)IL12(+) T-cells did not differ among the three groups (FIG. 6F).

### Example 7

### Adoptive Transfer of CD8(+) T-Cells from p210 Immunized Mice to Naive Recipients Recapitulates the Athero-Protective Effect of p210 Immunization

Donor apoE(−/−) mice were subjected to the same immunization protocol with the same groupings, namely: PBS, cBSA/alum, or p210/cBSA/alum. Recipient naïve male apoE(−/−) mice were injected with donor cells at 6-7 weeks of age and were fed normal chow until 13 weeks of age when chow was switched to high cholesterol diet until euthanasia at 25 weeks of age.

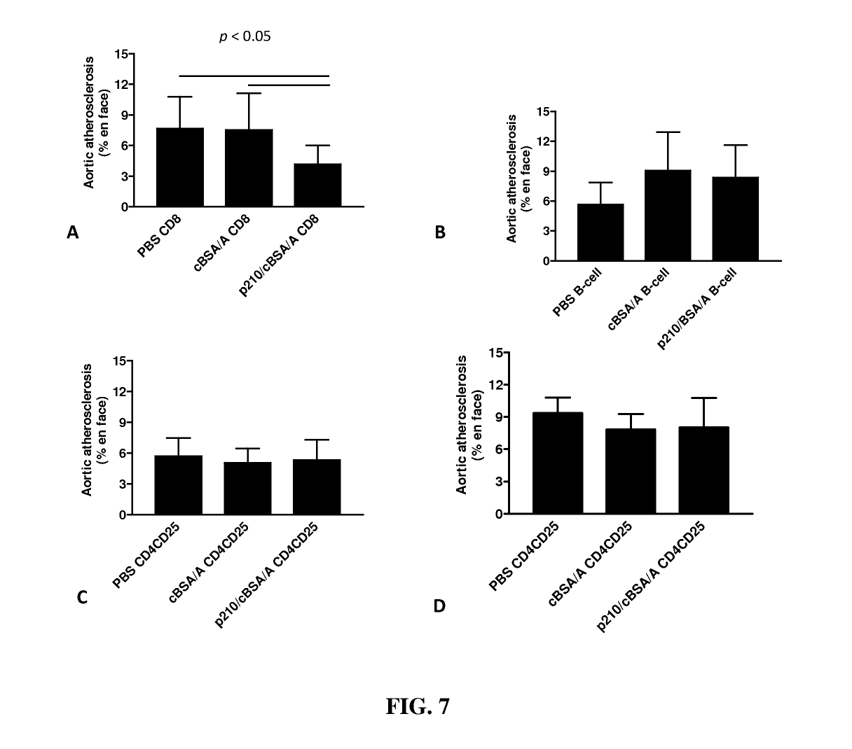

At euthanasia, the recipient mice injected with CD8(+) T-cells from p210/cBSA/alum group developed significantly less atherosclerotic lesions in aorta compared to the recipient mice injected with CD8(+) T-cells from PBS or cBSA/alum groups, strongly suggesting that the effector T cell induced by the vaccine are CD8⁺ and is mechanistically involved (FIG. 7A).

This reduction of aortic lesions was coupled with decreased splenic CD11c(+) DCs (PBS group: 4.3±1.7%; cBSA/alum group: 3.4±0.3%; p210/cBSA/alum group: 1.5±0.3%; n=5 each group, p<0.05 p210/cBSA/alum group vs. PBS or cBSA/alum group by ANOVA) with no difference in circulating levels of total cholesterol among 3 groups (PBS group: 1083±296 mg/dl; cBSA/alum group: 975±401 mg/dl; p210/cBSA/alum group: 1098±379 mg/dl).

Adoptive transfer of B cells isolated from the spleens of p210 immunized donor mice did not affect atherosclerosis in recipient mice compared to mice receiving B cells from other donors (FIG. 7B) These observations ruled out B cells as mediators of athero-protective effect of p210 immunization.

To rule out CD4(+)CD25(+) T-cells as possible athero-protective mediators induced by sub-cutaneous p210 immunization, Applicants adoptively transferred CD4(+)CD25(+)T-cells at a dose of 1×10⁵ cells/mouse into naïve recipient apoE−/− mice. There was no difference in lesion size among the 3 groups of CD4(+)CD25(+)T-cell recipients. Depletion of CD25⁺ cells from the pool of CD8⁺ T cells abrogated the reduction in atherosclerosis observed in the p210/cBSA/alum recipient mice, further supporting the notion that CD8⁺ CD25⁺ T cells are mechanistically involved in the protective effects of the vaccine against atherosclerosis (FIG. 7C). Transfer of a higher number of CD4(+)CD25(+) T-cells at 3×10⁵ cells/mouse did not reduce lesion sizes in all 3 recipient groups (FIG. 7D).

### Example 8

### Increased Cytolytic Activity of CD8(+) T Cells from p210 Immunized Mice Against Dendritic Cells In Vitro

Given the observation that p210 immunization reduced DCs in the immunization sites and atherosclerotic plaques and adoptive transfer of CD8(+) T-cells from p210 immunized donors rendered a decrease of splenic DCs in the recipients, Applicants hypothesized that DCs could be a potential target of CD8(+) T-cells.

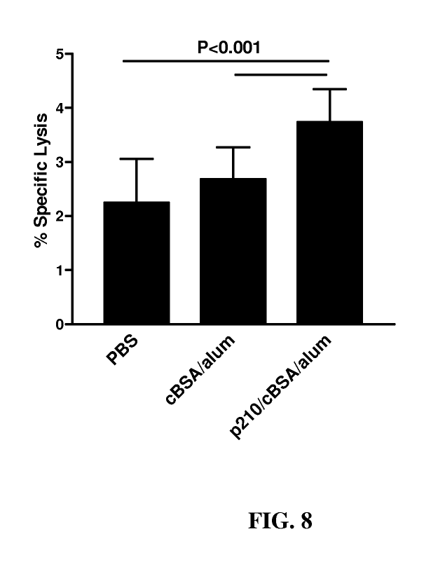

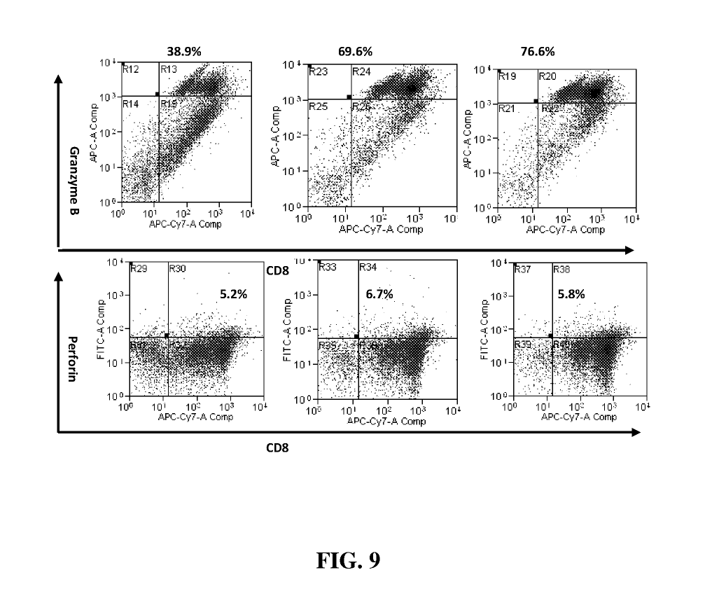

To test this, Applicants co-cultured bone marrow derived DCs with CD8(+) T-cells from various immunized groups. CD8(+) T-cells from p210 immunized mice significantly increased the percentage of DC death when compared to those from PBS or BSA/alum groups (FIG. 8). This increased cytolytic function of CD8(+) T-cells was associated with increased granzyme B expression but not perforin (FIG. 9).

### Example 9

### Immunization with p210 does not Affect the Adaptive Immune Response to Other T-Cell Dependent or Independent Antigens

Given the observations that p210 immunization decreased CD11c(+) DCs and reduced adaptive IgG response to p210, Applicants next tested if such modulation of DCs by p210 immunization would alter the host immune response to other antigens.

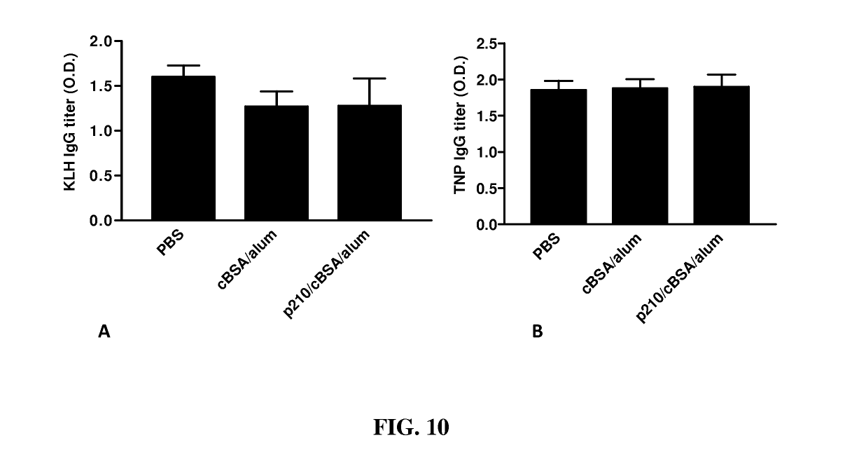

Applicants first immunized mice with p210 as described in previous sections followed by two separate subcutaneous KLH immunizations or intra-peritoneal injection of TNP-LPS. Using the KLH- or TNP-IgG titer as a surrogate for the efficacy of individual immunization, Applicants found that there was no difference in KLH- or TNP-IgG titers between p210 immunized mice and the titers from mice of PBS or cBSA/alum groups (FIG. 10).

### Example 10

### ApoB-100 Related Peptide P210 Immunization Reduces Blood Pressure Induced by Angiotensin

Male apoE KO mice were subcutaneously immunized at 7, 10, and 12 weeks of age with 100 μg of either Group 1: P210/cBSA conjugate using alum as adjuvant (P210); Group 2: control-100 μg of cBSA/alum (cBSA); Group 3: control PBS (PBS). 14 P210, 17 cBSA, 16 PBS, and 8 Saline injected mice were examined.

AngII (1000 ng/Kg/min) was delivered by a subcutaneous osmotic pump implanted at 10 weeks of age for 4 weeks to cause an increase in blood pressure in all three groups. Saline was delivered to the control group. Mice were sacrificed at 14 weeks of age of age. The mice were fed normal chow for the duration of the experiment.

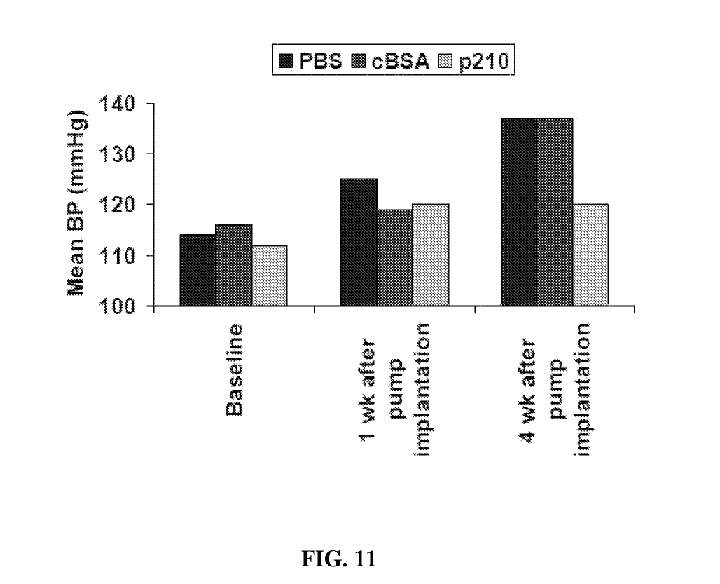

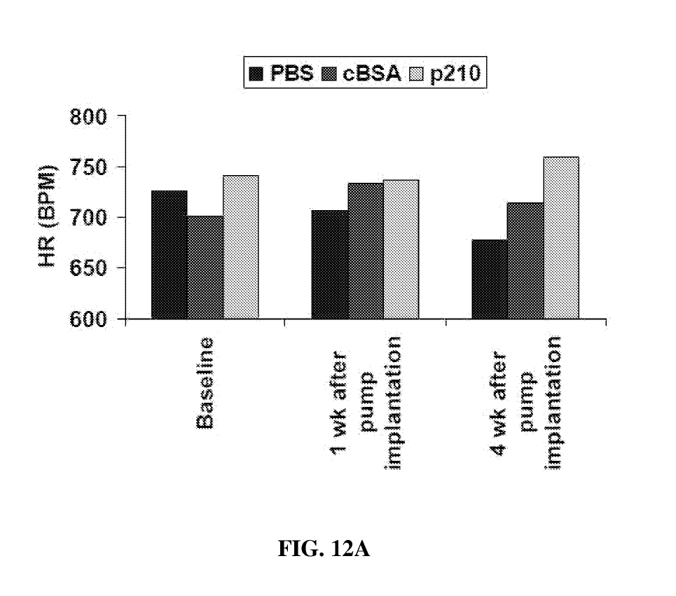

FIG. 11 shows an approximate 11% reduction in blood pressure in P210 vaccinated mice 4 weeks after pump implantation with a concomitant approximate 7% change in hearth rate in P210 vaccinated mice 4 weeks after pump implantation (FIG. 12A). FIG. 12B shows the time course of mean blood pressure change throughout the duration of experiments. Mice received treatment (PBS, cBSA/alum or p210/cBSA/alum) at 7, 10 and 12 weeks of age. Angiotensin II infusion via implanted osmotic pump was started at 10 weeks of age. Mice were euthanized at 14 weeks of age. Blood pressure was measured throughout the duration of experiment. Mean blood pressure gradually increased after angiotensin II infusion was started. At 13 weeks of age, mice immunized with p210/cBSA/alum had a significantly lower mean blood pressure when compared to that in the other 2 groups.

According to the above data it is expected that a p210 vaccine can prevent HTN.

A possible mechanism of action provided herein for guidance purposes only and not intended to be limiting is that p210 immunization reduces BP; and that the effect of p210 immunization is mediated by CD8 to a same or comparable extent detected for reduction of atherosclerosis illustrated in the following examples. Accordingly, ability to elicit a T cell response is specific for p210 (antigen specificity) and other apoB-100 peptides are expected to show similar antigen-specific CD8 effect.

A further possible mechanism of action provided herein for guidance purposes only and not intended to be limiting is that p210 action is performed also through modulation of angiotensin expression. Based on published anti-HTN vaccine literature, an anti-angiotensin vaccine can treat HTN. As a consequence, based on anti-angiotensin vaccine, multiple administration can be desired in certain condition and for certain types of individuals.

### Example 11

### Immunization with an apoB-100 Immunogenic Fragments Reduces Hypertension and Mortality in Angiotensin II-Induced Aortic Aneurysm

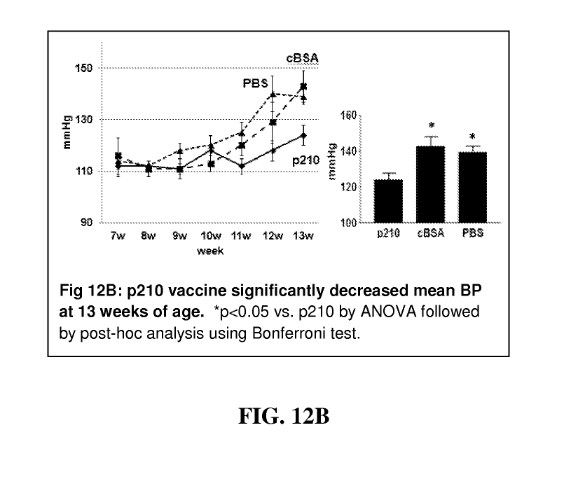

ApoE (−/−) mice were immunized with p210/cBSA/Alum (p210; 100 μg) at 7, 10, and 12 weeks of age. Mice receiving PBS or cBSA/Alum (cBSA) served as controls. At 10 weeks of age, mice were subcutaneously implanted with an osmotic pump which released AngII (1 mg/Kg/min), and were euthanized 4 weeks later. The aorta, spleen, and lymph nodes (LN) were harvested. The p210 vaccine significantly reduced mortality due to AA rupture compared to controls (see FIG. 13).

Flow cytometric analysis of dendritic cells (DCs) in LNs and spleen showed intracellular IFN-γ expression was upregulated in the p210 group. Aortic superoxide production measured by in situ dihydroethidine method and aortic AT1 receptor (AT1R) expression measured by Western blot were significantly decreased in p210 group. The p210 vaccine significantly decreased mean arterial BP at 13 weeks of age (see Table 7).

Mortality from AngII induced AA rupture was significantly reduced by the p210 vaccine. This protective effect was associated with upregulation of IFN-γ expression in DCs and decreased arterial BP, AT1R expression, and superoxide production in aorta. The vaccine may be a promising new non-invasive treatment for AA.

TABLE 7
Flow cytometric analysis of intracellular IFN-γ
expression of dendritic cells (DCs)

| | p210 | cBSA | PBS |
|---|---|---|---|
| Spleen CD11c⁺CD86⁺IFN-γ⁺ | 19.5 ± 1.6* | 13.9 ± 1.4 | 15.3 ± 0.7 |
| DCs (N = 8 each) | | | |
| LN CD11c⁺CD86⁺IFN-γ⁺ DCs | 26.7 ± 1.6* | 17.7 ± 2.3 | 18.1 ± 2.4 |
| (N = 6 each) | | | |
| Aortic AT1R (N = 6 each) | 1.0 ± 0.2* | 3.1 ± 0.6 | 3.2 ± 0.5 |
| Aortic superoxide production | 1.1 ± 0.1* | 1.9 ± 0.2 | 1.6 ± 0.1 |
| (N = 9 each) | | | |
| Mean Arterial Blood Pressure | 124 ± 4* | 143 ± 6 | 139 ± 3 |
| (BP) | | | |

Spleen and LN DC values are percentage ± SEM of CD11c-gated cells.
AT1R values are arbitrary densitometric unit ± SEM.
Superoxide values are arbitrary fluorescent intensity unit ± SEM.
Mean BP values are mmHg ± SEM at 13 weeks of age; number of mice: p210 N = 9; cBSA N = 7; PBS N = 10.
*p < 0.05 vs cBSA and PBS control; ANOVA, followed by post-hoc test.

### Example 12

### Increased Cytolytic Activity of CD8 (+) T Cells from apoB-100 Immunogenic Fragments Immunized Mice is Specific to Lipid-Associated Antigens

Applicants have shown that immunization with apoB-100 related-peptide p210 significantly reduces atherosclerosis and decreases intra-plaque CD11c⁺ dendritic cells (DCs) in apoE−/− mice. Adoptive transfer experiments showed that athero-protection was mediated by CD8⁺ T cells. Because apoB-100 is found on the LDL fraction of serum lipids, Applicants assessed the CD8⁺ T cell cytolytic activity of p210 immunized mice specific to lipid-associated antigens presented by DCs.

ApoE−/− mice were immunized at 7, 9, and 12 weeks of age with p210/cBSA/alum, cBSA/alum, or PBS. One week after the third immunization, mice were euthanized to collect spleen CD8⁺ T cells. Bone-marrow derived DCs were differentiated from naïve apoE−/− mice and used as target cells. A four-hour lytic assay was performed using a CD8-to-DC ratio of 3:1 in culture medium with 10% FBS. The cells were then collected and stained for CD11c to identify DCs and 7-AAD to assess cell lysis using flow cytometry. There was significantly more lytic activity by CD8⁺ T cells from p210/cBSA/alum immunized mice compared to cBSA/alum and PBS (Table). When the assay was performed in media with delipidated FBS, the lytic activity specific to CD8⁺ T cells from p210/cBSA/alum immunized mice was abrogated (Table 8), suggesting that the lipid fraction of FBS in the culture media provided a source of antigen. Loading of DCs with FITC-labeled p210 24 hours prior to the lytic assay demonstrated antigen uptake and specificity of the lytic activity of CD8⁺ T cells from p210/cBSA/alum immunized mice (see Table 8).

These results show that the cytolytic function of CD8⁺ T cells targeting DCs are specific to lipid-associated antigens, specifically the p210 fragment of apoB-100, and this may underly the protective effects of p210 immunization.

TABLE 8
Flow cytometric analysis of cytolytic activity of CD8 (+) T cells.

| | p210/cBSA/alum | cBSA/alum | PBS |
|---|---|---|---|
| Normal medium (N = | 3.7 ± 0.6* | 2.7 ± 0.6 | 2.3 ± 0.8 |
| 11 each) | | | |
| Delipidated medium (N = | 2.3 ± 0.4 | 2.4 ± 0.8 | 2.5 ± 0.5 |
| 5 each) | | | |
| FITC-p210 loaded (N = | 10.4 ± 0.1† | 7.3 ± 0.4 | 7.8 ± 1.2 |
| 3 each) | | | |

All flow cytometric analysis performed on CD11c-gated cells.
CD11c-gated FITC⁺ cells only were assessed in FITC-p210 loaded assay.
Values are percent lysis relative to basal lysis.
*P < 0.001;
†P < 0.01 by ANOVA.

### Example 13

### Antibody Response to the p210 Vaccine

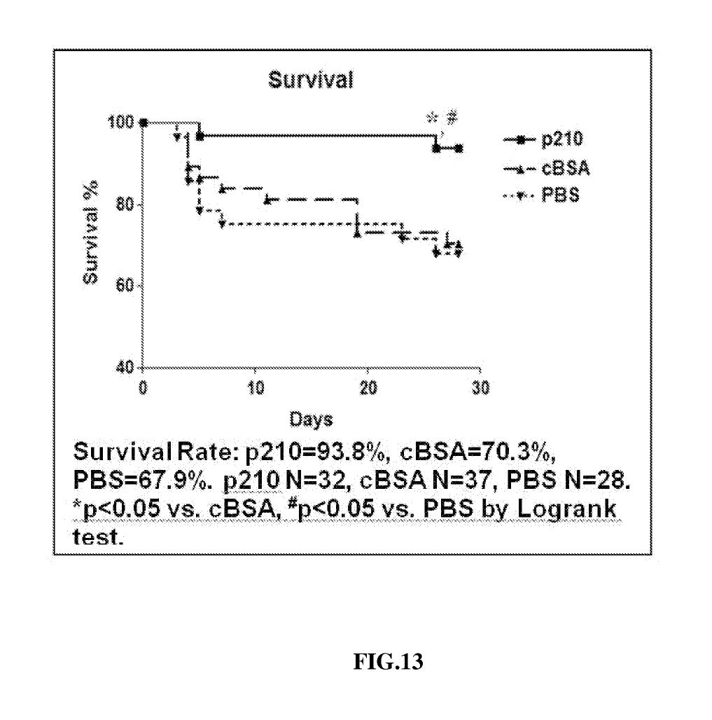

Antibody titers to p210 was low prior to immunization. At euthanasia at 25 weeks of age, there was a significant increase in p210 IgM titer in all groups (FIG. 14), suggesting an endogenous immune response against self-peptide p210. There was a significant increase in p210 IgG titers in both cBSA/alum group and p210/cBSA/alum compared with the PBS group, but titers in the cBSA/alum was surprisingly the higher between the 2 responding groups. The presence of alum as adjuvant in the cBSA/alum group and p210/cBSA/alum groups likely resulted in class switching of the IgM response to IgG, which did not occur in the PBS group.

### Example 14

### CD4 (+) T cell and CD8 (+) T Cell Response to the p210 Vaccine

T cells from superficial cervical and axillary lymph nodes (LN) from mice one week after primary immunization were collected to assess the T cell immune response. CD4⁺ CD25⁺ T cells in the lymph nodes (Table 1) did not differ among 3 groups. Splenic CD4⁺ CD25⁺IL-10⁺ T cell population significantly increased in the cBSA/alum group. However, this increased response was significantly attenuated by the p210/cBSA/alum immunization (Table 9). Interestingly, splenic CD4⁺ CD62L⁺ T cell (Table 1) population was lower in cBSA/alum group.

One week after primary immunization, the CD8⁺ CD25⁺ T cell population in the lymph nodes was significantly higher in p210/cBSA/alum group when compared to that of PBS or cBSA/alum groups (Table 2). There was a significantly larger population of splenic CD8⁺ CD25⁺IL-10⁺ T cells in p210/cBSA/alum group when compared to PBS or cBSA/alum groups (Table 2). The splenic CD8⁺ CD62L⁺ T cell population was significantly higher in p210/cBSA/alum group when compared to that of PBS or cBSA/alum groups (Table 9). The T cell profile at other time points were not significantly different between groups.

TABLE 9
CD4 (+) and CD8 (+) T cell response to the p210 vaccine

| | | |
|---|---|---|
| PBS | cBSA/alum | p210/cBSA/alum |

CD4⁺ T cell response to p210 vaccine.

| | | | |
|---|---|---|---|
| LN CD4+CD25+ | 12.9 ± 1.9 | 12.5 ± 1.4 | 14.0 ± 2.8 |
| Spl CD4+CD25+IL-10+ | 2.3 ± 0.3 | 4.3 ± 2.1* | 1.7 ± 0.6 |
| Spl CD4+CD62L+ | 26.7 ± 1.7 | 21.4 ± 2.7* | 29.9 ± 4.8 |

CD8⁺ T cell response to p210 vaccine.

| | | | |
|---|---|---|---|
| LN CD8+CD25+ | 4.4 ± 0.8 | 4.1 ± 1.0 | 6.8 ± 3.0* |
| Spl CD8+CD25+IL-10+ | 4.9 ± 3.9 | 6.0 ± 3.2 | 12.6 ± 3.9* |
| Spl CD8+CD62L+ | 18.4 ± 3.4 | 19.0 ± 5.5 | 27.6 ± 5.1* |

P < 0.05 vs. other groups

### Example 15

### Effector Role of CD8⁺ CD25⁺ T Cells Involves Cytotoxic Function

The vaccine reduced DC presence in the plaques (FIG. 3), and in the spleens of p210/cBSA/alum recipient mice, suggesting that the effector role of CD8⁺ T cells after immunization was manifested in decreasing DCs in the plaque. Applicants therefore assessed the effect of the vaccine on cytotoxic activity of CD8⁺ T cells against syngeneic bone marrow-derived DCs. CD8⁺ T cells from the immunized groups were negatively isolated using a CD8 selection Dynabeads kit (Invitrogen) followed by co-culture with DCs in a CD8:DC ratio of 3:1 in RPMI supplemented with 10% FBS. Cells were collected and processed for flow cytometric determination of CD11c⁺ and 7-AAD 4 hours later.²⁰ Dendritic cell death without CD8⁺ T cells in the co-culture was used as baseline and percentage of specific lysis of cells was calculated using a method described previously.²⁰

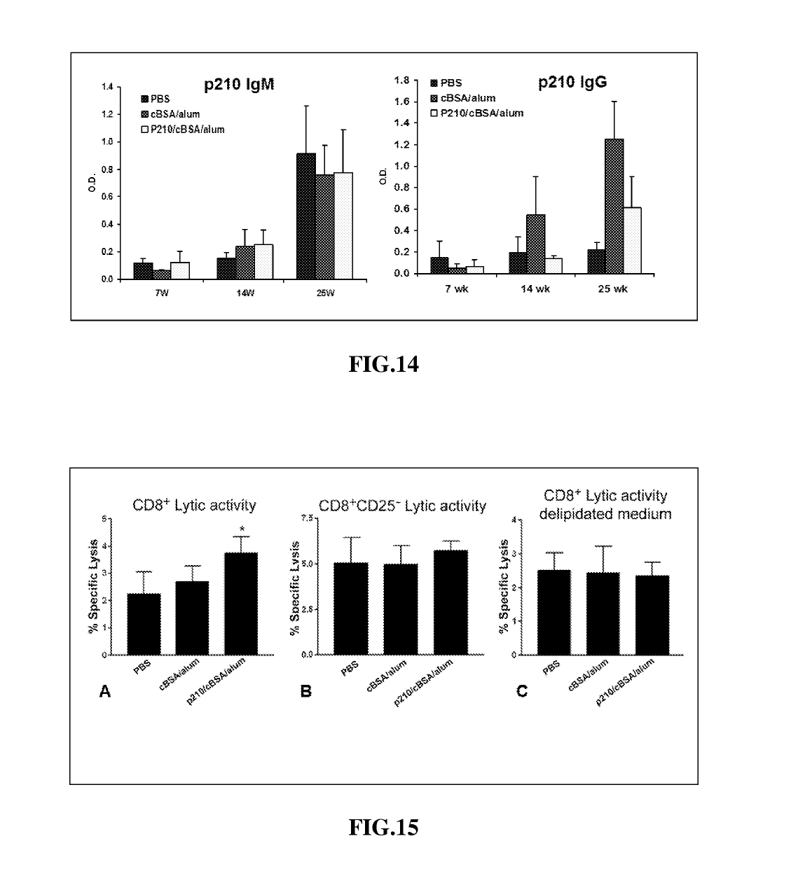

CD8⁺ T cells from p210 immunized mice significantly increased the percentage of DC lysis when compared to those from PBS or cBSA/alum groups (FIG. 15, panel A). This increased cytolytic function of CD8⁺ T cells was associated with increased granzyme B expression but not perforin. Depletion of CD25⁺ cells abrogated the increased cytolytic activity specific to the CD8⁺ T cells from p210 immunized mice (FIG. 15, panel B) indicating that CD8⁺ CD25⁺ T cells were the effector population. The increased cytolytic function specific to CD8⁺ T cells from p210 immunized mice was also lost with the use of delipidated serum supplemented medium (FIG. 15, panel C), indicating that the antigen on the target DCs recognized by the CTLs was derived from serum LDL containing apoB-100 in the medium.

### Example 16

### p210 Peptide is Endocytosed by DCs In Vitro

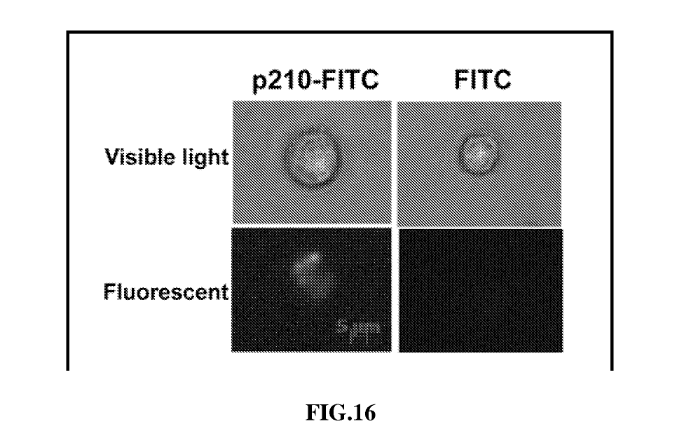

Peptide loading on BMDCs was defined using p210 labeled with FITC (FITC conjugating kit from Pierce). The presence of FITC fluorescence in the dendritic cells indicated uptake of p210 by dendritic cells (FIG. 16).

### Example 17

### p210 Peptide is Presented by DCs to CD8+ T Cells

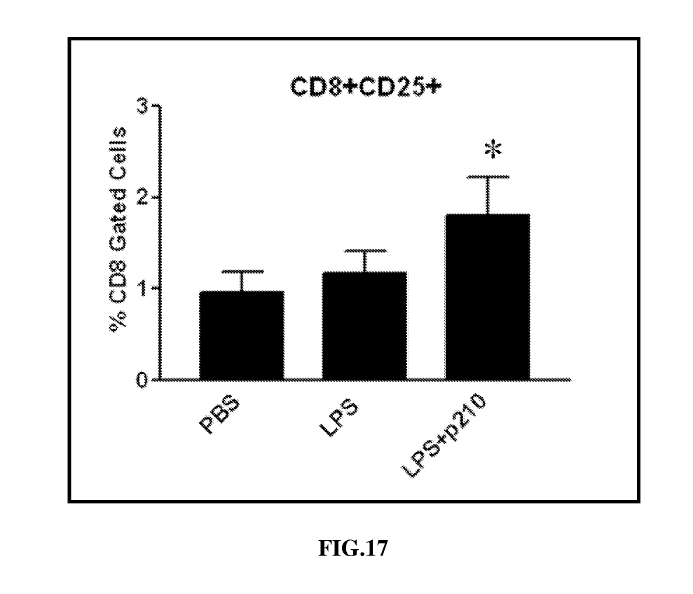

The p210 peptide contains the proteoglycan binding site of the apoB-100 molecule. This peptide is a cell-penetrating peptide capable of efficiently delivering antigens for cross-presentation to cytotoxic CD8⁺ T cells.⁵³ Applicants therefore assessed activation of CD8⁺ CD25⁻ T cells co-cultured with DCs loaded with p210 and matured with LPS. There was significantly increased CD8⁺ CD25⁺ T cells 48 hours after co-culture with p210-loaded DCs treated with LPS compared to untreated, or LPS only treated co-cultures (FIG. 17). The results suggest that the p210 antigen is presented by DCs to CD8⁺ T cells.

### Example 18

### p210-Loaded DCs are Specifically Targeted by Immune CD8⁺ T Cells

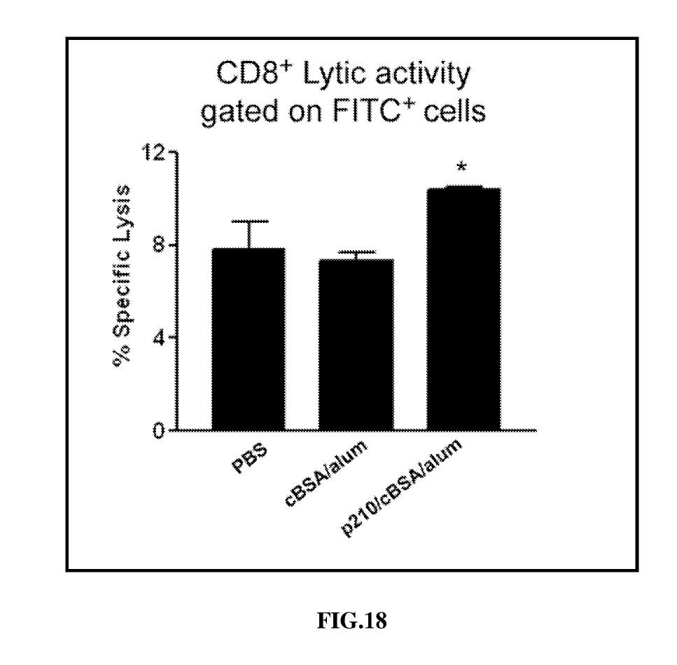

The results shown above in Example 17 support the notion that p210 is presented by DCs to CD8⁺ T cells. It remained unclear if the lytic activity against DCs was specific to the p210 antigen. Applicants therefore repeated the lytic assay using FITC-labeled p210 loaded BMDC as targets. Lytic activity against FITC DCs was significantly increased in CD8⁺ T cells from the p210/cBSA/alum mice (FIG. 18), indicating antigen specific lytic activity.

In summary, in several embodiments herein described are immunostimulatory agents, T cell, compositions, methods and systems for treating and/or preventing various conditions in a individual and in particular in a human individual.

The examples set forth above are provided to give those of ordinary skill in the art a complete disclosure and description of how to make and use the embodiments of the molecules, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence. Further, the sequence listing submitted herewith in the txt file “P700-PCT-2011-11-11-Sequence Listing_ST25” created on Nov. 11, 2011 forms an integral part of the present application and is incorporated herein by reference in its entirety.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### REFERENCES

1. Shah, P. K., K. Y. Chyu, G. N. Fredrikson, and J. Nilsson. 2005. Immunomodulation of atherosclerosis with a vaccine. Nat. Clin. Pract. Cardiovasc. Med. 2:639-646
2. Hansson, G. K., P. Libby, U. Schonbeck, and Z. Q. Yan. 2002. Innate and adaptive immunity in the pathogenesis of atherosclerosis. Circ. Res. 91:281-291
3. Chyu, K. Y., X. Zhao, O. S. Reyes, S. M. Babbidge, P. C. Dimayuga, J. Yano, B. Cercek, G. N. Fredrikson, J. Nilsson, and P. K. Shah. 2005. Immunization using an Apo B-100 related epitope reduces atherosclerosis and plaque inflammation in hypercholesterolemic apo E (−/−) mice. Biochem. Biophys. Res. Commun. 338:1982-1989
4. Fredrikson, G. N., I. Soderberg, M. Lindholm, P. Dimayuga, K. Y. Chyu, P. K. Shah, and J. Nilsson. 2003. Inhibition of Atherosclerosis in ApoE-Null Mice by Immunization with ApoB-100 Peptide Sequences. Arterioscler. Thromb. Vasc. Biol. 23:879-884
5. Fredrikson, G. N., L. Andersson, I. Soderberg, P. Dimayuga, K. Y. Chyu, P. K. Shah, and J. Nilsson. 2005. Atheroprotective immunization with MDA-modified apo B-100 peptide sequences is associated with activation of Th2 specific antibody expression. Autoimmunity 38:171-179
6. Fredrikson, G. N., H. Bjorkbacka, I. Soderberg, I. Ljungcrantz, and J. Nilsson. 2008. Treatment with apo B peptide vaccines inhibits atherosclerosis in human apo B-100 transgenic mice without inducing an increase in peptide-specific antibodies. J. Intern. Med. 1-8
7. Klingenberg, R., M. Lebens, A. Hermansson, G. N. Fredrikson, D. Strodthoff, M. Rudling, D. F. Ketelhuth, N. Gerdes, J. Holmgren, J. Nilsson, and G. K. Hansson. 2010. Intranasal Immunization With an Apolipoprotein B-100 Fusion Protein Induces Antigen-Specific Regulatory T Cells and Reduces Atherosclerosis. Arterioscler. Thromb. Vasc. Biol. 30:946-952
8. Fredrikson, G. N., B. Hedblad, G. Berglund, R. Alm, M. Ares, B. Cercek, K. Y. Chyu, P. K. Shah, and J. Nilsson. 2003. Identification of Immune Responses Against Aldehyde-Modified Peptide Sequences in ApoB Associated With Cardiovascular Disease. Arterioscler. Thromb. Vasc. Biol. 23:872-878
9. Schiopu, A., J. Bengtsson, I. Soderberg, S. Janciauskiene, S. Lindgren, M. P. Ares, P. K. Shah, R. Carlsson, J. Nilsson, and G. N. Fredrikson. 2004. Recombinant Human Antibodies Against Aldehyde-Modified Apolipoprotein B-100 Peptide Sequences Inhibit Atherosclerosis. Circulation 2004. 110:2047-2052
10. Sjogren, P., G. N. Fredrikson, A. Samnegard, C. G. Ericsson, J. Ohrvik, R. M. Fisher, J. Nilsson, and A. Hamsten. 2008. High plasma concentrations of autoantibodies against native peptide 210 of apoB-100 are related to less coronary atherosclerosis and lower risk of myocardial infarction. Eur. Heart J. 29:2218-2226
11. Dimayuga, P., B. Cercek, S. Oguchi, G. N. Fredrikson, J. Yano, P. K. Shah, S. Jovinge, and J. Nilsson. 2002. Inhibitory effect on arterial injury-induced neointimal formation by adoptive B-cell transfer in Rag-1 knockout mice. Arterioscler. Thromb. Vasc. Biol. 22:644-649
12. Caligiuri, G., A. Nicoletti, B. Poirier, and G. K. Hansson. 2002. Protective immunity against atherosclerosis carried by B cells of hypercholesterolemic mice. J. Clin. Invest. 109:745-753
13. Yang, K., D. Li, M. Luo, and Y. Hu. 2006. Generation of HSP60-specific regulatory T cell and effect on atherosclerosis. Cell Immunol. 243:90-95
14. Mor, A., D. Planer, G. Luboshits, A. Afek, S. Metzger, T. Chajek-Shaul, G. Keren, and J. George. 2007. Role of naturally occurring CD4+ CD25+ regulatory T cells in experimental atherosclerosis. Arterioscler. Thromb. Vasc. Biol. 27:893-900
15. Ait-Oufella, H., B. L. Salomon, S. Potteaux, A. K. Robertson, P. Gourdy, J. Zoll, R. Merval, B. Esposito, J. L. Cohen, S. Fisson, R. A. Flavell, G. K. Hansson, D. Klatzmann, A. Tedgui, and Z. Mallat. 2006. Natural regulatory T cells control the development of atherosclerosis in mice. Nat. Med. 12:178-180
16. Yang, G. X., Z. X. Lian, Y. H. Chuang, Y. Moritoki, R. Y. Lan, K. Wakabayashi, A. A. Ansari, R. A. Flavell, W. M. Ridgway, R. L. Coppel, K. Tsuneyama, I. R. Mackay, and M. E. Gershwin. 2008. Adoptive transfer of CD8(+) T cells from transforming growth factor beta receptor type II (dominant negative form) induces autoimmune cholangitis in mice. Hepatology. 47:1974-1982
17. Zhou, X., A. Nicoletti, R. Elhage, and G. K. Hansson. 2000. Transfer of CD4(+) T cells aggravates atherosclerosis in immunodeficient apolipoprotein E knockout mice. Circulation 102:2919-2922
18. Zhou, X., A. K. Robertson, C. Hjerpe, and G. K. Hansson. 2006. Adoptive transfer of CD4+ T cells reactive to modified low-density lipoprotein aggravates atherosclerosis. Arterioscler. Thromb. Vasc. Biol. 26:864-870
19. Inaba, K., M. Inaba, N. Romani, H. Aya, M. Deguchi, S. Ikehara, S. Muramatsu, and R. M. Steinman. 1992. Generation of large numbers of dendritic cells from mouse bone marrow cultures supplemented with granulocyte/macrophage colony-stimulating factor. J. Exp. Med. 176:1693-1702
20. Lecoeur, H., M. Fevrier, S. Garcia, Y. Riviere, and M. L. Gougeon. 2001. A novel flow cytometric assay for quantitation and multiparametric characterization of cell-mediated cytotoxicity. J. Immunol. Methods 253:177-187
21. Palinski, W., E. Miller, and J. L. Witztum. 1995. Immunization of low density lipoprotein (LDL) receptor-deficient rabbits with homologous malondialdehyde-modified LDL reduces atherogenesis. Proc. Natl. Acad. Sci. U.S. A 92:821-825
22. Ameli, S., A. Hultgardh-Nilsson, J. Regnstrom, F. Calara, J. Yano, B. Cercek, P. K. Shah, and J. Nilsson. 1996. Effect of immunization with homologous LDL and oxidized LDL on early atherosclerosis in hypercholesterolemic rabbits. Arterioscler. Thromb. Vasc. Biol. 16:1074-1079
23. Freigang, S., S. Horkko, E. Miller, J. L. Witztum, and W. Palinski. 1998. Immunization of LDL receptor-deficient mice with homologous malondialdehyde-modified and native LDL reduces progression of atherosclerosis by mechanisms other than induction of high titers of antibodies to oxidative neoepitopes. Arterioscler. Thromb. Vasc. Biol. 18:1972-1982
24. George, J., A. Afek, B. Gilburd, H. Levkovitz, A. Shaish, I. Goldberg, Y. Kopolovic, G. Wick, Y. Shoenfeld, and D. Harats. 1998. Hyperimmunization of apo-E-deficient mice with homologous malondialdehyde low-density lipoprotein suppresses early atherogenesis. Atherosclerosis 138:147-152
25. Zhou, X., G. Caligiuri, A. Hamsten, A. K. Lefvert, and G. K. Hansson. 2001. LDL immunization induces T-cell-dependent antibody formation and protection against atherosclerosis. Arterioscler. Thromb. Vasc. Biol. 21:108-114
26. Chyu, K. Y., 0. S. Reyes, X. Zhao, J. Yano, P. Dimayuga, J. Nilsson, B. Cercek, and P. K. Shah. 2004. Timing affects the efficacy of LDL immunization on atherosclerotic lesions in apo E (−/−) mice. Atherosclerosis 176:27-35
27. Zhou, X., A. K. Robertson, M. Rudling, P. Parini, and G. K. Hansson. 2005. Lesion development and response to immunization reveal a complex role for CD4 in atherosclerosis. Circ. Res. 96:427-434
28. Roselaar, S. E., P. X. Kakkanathu, and A. Daugherty. 1996. Lymphocyte populations in atherosclerotic lesions of apoE −/− and LDL receptor −/− mice. Decreasing density with disease progression. Arterioscler. Thromb. Vasc. Biol. 16:1013-1018
29. Zhou, X., S. Stemme, and G. K. Hansson. 1996. Evidence for a local immune response in atherosclerosis. CD4+ T cells infiltrate lesions of apolipoprotein-E-deficient mice. Am. J. Pathol. 149:359-366
30. Fyfe, A. I., J. H. Qiao, and A. J. Lusis. 1994. Immune-deficient mice develop typical atherosclerotic fatty streaks when fed an atherogenic diet. J. Clin. Invest. 94:2516-2520
31. Bobryshev, Y. V., T. Taksir, R. S. Lord, and M. W. Freeman. 2001. Evidence that dendritic cells infiltrate atherosclerotic lesions in apolipoprotein E-deficient mice. Histol. Histopathol. 16:801-808
32. Niessner, A., and C. M. Weyand. 2009. Dendritic cells in atherosclerotic disease. Clin. Immunol. 134:25-32
33. Paulson, K. E., S. N. Zhu, M. Chen, S. Nurmohamed, J. Jongstra-Bilen, and M. I. Cybulsky. 2010. Resident intimal dendritic cells accumulate lipid and contribute to the initiation of atherosclerosis. Circ. Res. 106:383-390
34. Liu, P., Y. R. Yu, J. A. Spencer, A. E. Johnson, C. T. Vallanat, A. M. Fong, C. Patterson, and D. D. Patel. 2008. CX3CR1 deficiency impairs dendritic cell accumulation in arterial intima and reduces atherosclerotic burden. Arterioscler. Thromb. Vasc. Biol. 28:243-250
35. Wu, H., R. M. Gower, H. Wang, X. Y. Perrard, R. Ma, D. C. Bullard, A. R. Burns, A. Paul, C. W. Smith, S. I. Simon, and C. M. Ballantyne. 2009. Functional role of CD11c+ monocytes in atherogenesis associated with hypercholesterolemia. Circulation. 119:2708-2717
36. Sakamoto, N., K. Tsuji, L. M. Muul, A. M. Lawler, E. F. Petricoin, F. Candotti, J. A. Metcalf, J. A. Tavel, H. C. Lane, W. J. Urba, B. A. Fox, A. Varki, J. K. Lunney, and A. S. Rosenberg. 2007. Bovine apolipoprotein B-100 is a dominant immunogen in therapeutic cell populations cultured in fetal calf serum in mice and humans. Blood 110:501-508
37. van den Elzen, p., S. Garg, L. Leon, M. Brigl, E. A. Leadbetter, J. E. Gumperz, C. C. Dascher, T. Y. Cheng, F. M. Sacks, P. A. Illarionov, G. S. Besra, S. C. Kent, D. B. Moody, and M. B. Brenner. 2005. Apolipoprotein-mediated pathways of lipid antigen presentation. Nature 437:906-910
38. Mitchell D M, Ravkov E V, Williams M A Distinct roles for IL-2 and IL-15 in the differentiation and survival of CD8+ effector and memory T cells. J. Immunol. 2010 Jun. 15; 184(12):6719-30. Epub 2010 May 14
39. Perret R, Ronchese F. Effector CD8+ T cells activated in vitro confer immediate and long-term tumor protection in vivo. Eur J. Immunol. 2008 October; 38(10):2886-95.
40. Kamimura D, Bevan M J. Naive CD8+ T cells differentiate into protective memory-like cells after IL-2 anti IL-2 complex treatment in vivo. J Exp Med. 2007 Aug. 6; 204(8):1803-12. Epub 2007 Jul. 30.
41. J. Immunol. 2006; 177:5868-5877
42. J. Immunol. 2004; 172:1991-1995
43. San-Hwan Chen et al The complete cDNA and amino acid sequence of Human Apolipoprotein B100 Journal of Biological Chemistry 1986 Vol. 261No 28, Issue of October 5, 12918-12921
44. Chou P Y, Fasman G O, Adv Enzymol Relat Areas Mol BioI. 1978; 47: 45-148. Prediction of the secondary structure of proteins from their amino acid sequence;
45. Margalit H, Spouge J L, Cornette J L, Cease K B, Delisi C, Berzofsky J A, J., Immunol. 1987 Apr. 1; 138(7):2213-29. Prediction of immunodominant helper T cell antigenic sites from the primary sequence;
46. Jameson B A, Wolf H., Division of Biology, California Institute of Technology, Pasadena, Calif. 91125, Comput Appl BioscL 1988 March; 4(1): 181-6. The antigenic index: a novel algorithm for predicting antigenic determinants;
47. Reyes V E, Lew R A, Lu S., Humphreys R E, Methods Enzymol. 1991; 202:22538. Prediction of alpha helices and T cell-presented sequences in proteins with algorithms based on strip-of-helix hydrophobicity index (SOHHI);
48. Maksyutov A Z, Zagrebelnaya E S, Comput Appl BioscL 1993 June; 9(3): 291-7. ADEPT: a computer program for prediction of protein antigenic determinants;
49. Pellequer J L, Westhof E., J Mol Graph. 1993 September; 11(3): 204-10, 1912. PREDITOP: a program for antigenicity prediction;
50. Lu et al., Tibtech, vol. 9, July 1991 pp. 238-242 Common Principles in Protein Folding and Antigen Protection; and
51. Laura Raddrizzani and Juergen Hammer BRIEFINGS IN BIOINFORMATICS. VOL I. NO 2. 179-189. MAY 2000 Epitope scanning usingvirtual Matrix-based algorithms
52. R. Wu, R. Giscombe, G. Holm & A. K. Lefvert “Induction of Human Cytotoxic T Lymphocytes by Oxidized Low Density Lipoproteins” Scand. J. Immunol. 43,381-384, 1996.
53. Sakamoto, N and Rosenberg, A S. Apolipoprotein B binding domains: evidence that they are cell-penetrating peptides that efficiently deliver antigenic peptide for cross-presentation of cytotoxic T cells. J. Immunol. 4-15-2011; 186:5004-5011.

1. A method to treat and/or prevent a condition treatable with an immunogenic fragment of ApoB-100 or an immunogenically active portion thereof in an human individual, the method comprising
administering to the individual an effective amount of at least one immunogenic fragment of ApoB-100 or the immunogenically active portion thereof, wherein the immunogenic fragment comprises one of SEQ ID NOs 1-302, wherein the effective amount is less than 1 mg, and wherein as a result of the administration, the condition treatable with an immunogenic fragment of ApoB-100 is treated and/or prevented.

2. The method of claim 1, wherein the immunogenic fragment is associated to atherosclerosis reduction.

3. (canceled)

4. The method of claim 1, wherein the one or more immunogenic fragments comprises one or more peptides each comprising one of SEQ ID NO: 1, SEQ ID NO: 2, SEQ ID NO:11, SEQ ID NO:25, SEQ ID NO:45, SEQ ID NO:74, SEQ ID NO:99, SEQ ID NO:100, SEQ ID NO:102, SEQ ID NO: 103, SEQ ID NO:105, SEQ ID NO:129, SEQ ID NO:143, SEQ ID NO:148, SEQ ID NO:210, or SEQ ID NO:301.

5. The method of claim 1, wherein the one or more immunogenic fragments comprises one or more peptides each comprising one of SEQ ID NO:2, SEQ ID NO:11, SEQ ID NO: 45, SEQ ID NO: 74, SEQ ID NO: 102, SEQ ID NO:148, SEQ ID NO:162, or SEQ ID NO:210.

6. The method of claim 1, wherein the one or more immunogenic fragments comprises a peptide having SEQ ID NO: 143 and a peptide having SEQ ID NO: 210.

7. The method of claim 1, wherein the one or more immunogenic fragments comprises a peptide having SEQ ID NO: 11, a peptide having SEQ ID NO: 25 and a peptide having SEQ ID NO: 74.

8. The method of claim 1, wherein the one or more immunogenic fragments comprises a peptide having SEQ ID NO: 2.

9. The method of claim 1, wherein the one or more immunogenic fragments comprise a peptide having SEQ ID NO: 45.

10. The method of claim 1, wherein the one or more immunogenic fragments comprise a peptide having SEQ ID NO: 210.

11. The method of claim 1, wherein the effective amount is about 100 μg to about 1000 μg.

12. The method of claim 1, wherein the effective amount is about 100 μg to about 500 μg, or about 500 μg to about less than a 1000 μg.

13. The method of claim 1, wherein the effective amount is about 100 μg to about 250 μg, or at least about 250 μg.

14. A pharmaceutical composition comprising less than 1 mg of one or more immunogenic fragments of ApoB-100 or an immunogenically active portion thereof and a pharmaceutically acceptable vehicle, wherein the immunogenic fragment comprises SEQ ID NOs:1-302.

15. The pharmaceutical composition of claim 14 wherein the composition comprises about 100 μg to about 1000 μg of the one or more immunogenic fragments of ApoB-100 or an immunogenically active portion thereof.

16. The pharmaceutical composition of claim 14 wherein the composition comprises about 1 μg to about 500 μg, or about 500 μg to about 1000 μg of the one or more immunogenic fragments of ApoB-100 or an immunogenically active portion thereof.

17. The pharmaceutical composition of claim 14 wherein the composition comprises about 100 μg to about 250 μg or at least about 250 μg of the one or more immunogenic fragments of ApoB-100 or an immunogenically active portion thereof.

18. A pharmaceutical composition comprising less than a 1 mg of one or more immunogenic fragments of ApoB-100 or an immunogenically active portion thereof and a pharmaceutically acceptable vehicle, wherein the immunogenic fragment consists of one of SEQ ID NOs:1-302.


SEQUENCE LISTING (302 sequences)

SEQ ID NO: 1 - protein, 20 aa, Artificial Sequence
  Synthetic peptide
EEEMLENVSLVCPKDATRFK

SEQ ID NO: 2 - protein, 20 aa, Artificial Sequence
  Synthetic peptide
ATRFKHLRKYTYNYEAESSS

SEQ ID NO: 3 - protein, 20 aa, Artificial Sequence
  Synthetic peptide
AESSSGVPGTADSRSATRIN

SEQ ID NO: 4 - protein, 20 aa, Artificial Sequence
  Synthetic peptide
ATRINCKVELEVPQLCSFIL

SEQ ID NO: 5 - protein, 20 aa, Artificial Sequence
  Synthetic peptide
CSFILKTSQCTLKEVYGFNP

SEQ ID NO: 6 - protein, 20 aa, Artificial Sequence
  Synthetic peptide
YGFNPEGKALLKKTKNSEEF

SEQ ID NO: 7 - protein, 20 aa, Artificial Sequence
  Synthetic peptide
NSEEFAAAMSRYELKLAIPE

SEQ ID NO: 8 - protein, 20 aa, Artificial Sequence
  Synthetic peptide
LAIPEGKQVFLYPEKDEPTY

SEQ ID NO: 9 - protein, 20 aa, Artificial Sequence
  Synthetic peptide
DEPTYILNIKRGIISALLVP

SEQ ID NO: 10 - protein, 20 aa, Artificial Sequence
  Synthetic peptide
ALLVPPETEEAKQVLFLDTV

SEQ ID NO: 11 - protein, 20 aa, Artificial Sequence
  Synthetic peptide
FLDTVYGNCSTHFTVKTRKG

SEQ ID NO: 12 - protein, 20 aa, Artificial Sequence
  Synthetic peptide
KTRKGNVATEISTERDLGQC

SEQ ID NO: 13 - protein, 20 aa, Artificial Sequence
  Synthetic peptide
DLGQCDRFKPIRTGISPLAL

SEQ ID NO: 14 - protein, 20 aa, Artificial Sequence
  Synthetic peptide
SPLALIKGMTRPLSTLISSS

SEQ ID NO: 15 - protein, 20 aa, Artificial Sequence
  Synthetic peptide
LISSSQSCQYTLDAKRKHVA

SEQ ID NO: 16 - protein, 20 aa, Artificial Sequence
  Synthetic peptide
RKHVAEAICKEQHLFLPFSY

SEQ ID NO: 17 - protein, 20 aa, Artificial Sequence
  Synthetic peptide
LPFSYNNKYGMVAQVTQTLK

SEQ ID NO: 18 - protein, 20 aa, Artificial Sequence
  Synthetic peptide
TQTLKLEDTPKINSRFFGEG

SEQ ID NO: 19 - protein, 20 aa, Artificial Sequence
  Synthetic peptide
FFGEGTKKMGLAFESTKSTS

SEQ ID NO: 20 - protein, 20 aa, Artificial Sequence
  Synthetic peptide
TKSTSPPKQAEAVLKTLQEL

SEQ ID NO: 21 - protein, 20 aa, Artificial Sequence
  Synthetic peptide
TLQELKKLTISEQNIQRANL

SEQ ID NO: 22 - protein, 20 aa, Artificial Sequence
  Synthetic peptide
QRANLFNKLVTELRGLSDEA

SEQ ID NO: 23 - protein, 20 aa, Artificial Sequence
  Synthetic peptide
LSDEAVTSLLPQLIEVSSPI

SEQ ID NO: 24 - protein, 20 aa, Artificial Sequence
  Synthetic peptide
VSSPITLQALVQCGQPQCST

SEQ ID NO: 25 - protein, 20 aa, Artificial Sequence
  Synthetic peptide
PQCSTHILQWLKRVHANPLL

SEQ ID NO: 26 - protein, 20 aa, Artificial Sequence
  Synthetic peptide
ANPLLIDVVTYLVALIPEPS

SEQ ID NO: 27 - protein, 20 aa, Artificial Sequence
  Synthetic peptide
IPEPSAQQLREIFNMARDQR

SEQ ID NO: 28 - protein, 20 aa, Artificial Sequence
  Synthetic peptide
ARDQRSRATLYALSHAVNNY

SEQ ID NO: 29 - protein, 20 aa, Artificial Sequence
  Synthetic peptide
AVNNYHKTNPTGTQELLDIA

SEQ ID NO: 30 - protein, 20 aa, Artificial Sequence
  Synthetic peptide
LLDIANYLMEQIQDDCTGDE

SEQ ID NO: 31 - protein, 20 aa, Artificial Sequence
  Synthetic peptide
CTGDEDYTYLILRVIGNMGQ

SEQ ID NO: 32 - protein, 20 aa, Artificial Sequence
  Synthetic peptide
GNMGQTMEQLTPELKSSILK

SEQ ID NO: 33 - protein, 20 aa, Artificial Sequence
  Synthetic peptide
SSILKCVQSTKPSLMIQKAA

SEQ ID NO: 34 - protein, 20 aa, Artificial Sequence
  Synthetic peptide
IQKAAIQALRKMEPKDKDQE

SEQ ID NO: 35 - protein, 20 aa, Artificial Sequence
  Synthetic peptide
DKDQEVLLQTFLDDASPGDK

SEQ ID NO: 36 - protein, 20 aa, Artificial Sequence
  Synthetic peptide
SPGDKRLAAYLMLMRSPSQA

SEQ ID NO: 37 - protein, 20 aa, Artificial Sequence
  Synthetic peptide
SPSQADINKIVQILPWEQNE

SEQ ID NO: 38 - protein, 20 aa, Artificial Sequence
  Synthetic peptide
WEQNEQVKNFVASHIANILN

SEQ ID NO: 39 - protein, 20 aa, Artificial Sequence
  Synthetic peptide
ANILNSEELDIQDLKKLVKE

SEQ ID NO: 40 - protein, 20 aa, Artificial Sequence
  Synthetic peptide
KLVKEALKESQLPTVMDFRK

SEQ ID NO: 41 - protein, 20 aa, Artificial Sequence
  Synthetic peptide
MDFRKFSRNYQLYKSVSLPS

SEQ ID NO: 42 - protein, 20 aa, Artificial Sequence
  Synthetic peptide
VSLPSLDPASAKIEGNLIFD

SEQ ID NO: 43 - protein, 20 aa, Artificial Sequence
  Synthetic peptide
NLIFDPNNYLPKESMLKTTL

SEQ ID NO: 44 - protein, 20 aa, Artificial Sequence
  Synthetic peptide
LKTTLTAFGFASADLIEIGL

SEQ ID NO: 45 - protein, 20 aa, Artificial Sequence
  Synthetic peptide
IEIGLEGKGFEPTLEALFGK

SEQ ID NO: 46 - protein, 20 aa, Artificial Sequence
  Synthetic peptide
ALFGKQGFFPDSVNKALYWV

SEQ ID NO: 47 - protein, 20 aa, Artificial Sequence
  Synthetic peptide
ALYWVNGQVPDGVSKVLVDH

SEQ ID NO: 48 - protein, 20 aa, Artificial Sequence
  Synthetic peptide
VLVDHFGYTKDDKHEQDMVN

SEQ ID NO: 49 - protein, 20 aa, Artificial Sequence
  Synthetic peptide
QDMVNGIMLSVEKLIKDLKS

SEQ ID NO: 50 - protein, 20 aa, Artificial Sequence
  Synthetic peptide
KDLKSKEVPEARAYLRILGE

SEQ ID NO: 51 - protein, 20 aa, Artificial Sequence
  Synthetic peptide
RILGEELGFASLHDLQLLGK

SEQ ID NO: 52 - protein, 20 aa, Artificial Sequence
  Synthetic peptide
QLLGKLLLMGARTLQGIPQM

SEQ ID NO: 53 - protein, 20 aa, Artificial Sequence
  Synthetic peptide
GIPQMIGEVIRKGSKNDFFL

SEQ ID NO: 54 - protein, 20 aa, Artificial Sequence
  Synthetic peptide
NDFFLHYIFMENAFELPTGA

SEQ ID NO: 55 - protein, 20 aa, Artificial Sequence
  Synthetic peptide
LPTGAGLQLQISSSGVIAPG

SEQ ID NO: 56 - protein, 20 aa, Artificial Sequence
  Synthetic peptide
VIAPGAKAGVKLEVANMQAE

SEQ ID NO: 57 - protein, 20 aa, Artificial Sequence
  Synthetic peptide
NMQAELVAKPSVSVEFVTNM

SEQ ID NO: 58 - protein, 20 aa, Artificial Sequence
  Synthetic peptide
FVTNMGIIIPDFARSGVQMN

SEQ ID NO: 59 - protein, 20 aa, Artificial Sequence
  Synthetic peptide
GVQMNTNFFHESGLEAHVAL

SEQ ID NO: 60 - protein, 20 aa, Artificial Sequence
  Synthetic peptide
AHVALKAGKLKFIIPSPKRP

SEQ ID NO: 61 - protein, 20 aa, Artificial Sequence
  Synthetic peptide
SPKRPVKLLSGGNTLHLVST

SEQ ID NO: 62 - protein, 20 aa, Artificial Sequence
  Synthetic peptide
HLVSTTKTEVIPPLIENRQS

SEQ ID NO: 63 - protein, 20 aa, Artificial Sequence
  Synthetic peptide
ENRQSWSVCKQVFPGLNYCT

SEQ ID NO: 64 - protein, 20 aa, Artificial Sequence
  Synthetic peptide
LNYCTSGAYSNASSTDSASY

SEQ ID NO: 65 - protein, 20 aa, Artificial Sequence
  Synthetic peptide
DSASYYPLTGDTRLELELRP

SEQ ID NO: 66 - protein, 20 aa, Artificial Sequence
  Synthetic peptide
LELRPTGEIEQYSVSATYEL

SEQ ID NO: 67 - protein, 20 aa, Artificial Sequence
  Synthetic peptide
ATYELQREDRALVDTLKFVT

SEQ ID NO: 68 - protein, 20 aa, Artificial Sequence
  Synthetic peptide
LKFVTQAEGAKQTEATMTFK

SEQ ID NO: 69 - protein, 20 aa, Artificial Sequence
  Synthetic peptide
TMTFKYNRQSMTLSSEVQIP

SEQ ID NO: 70 - protein, 20 aa, Artificial Sequence
  Synthetic peptide
EVQIPDFDVDLGTILRVNDE

SEQ ID NO: 71 - protein, 20 aa, Artificial Sequence
  Synthetic peptide
RVNDESTEGKTSYRLTLDIQ

SEQ ID NO: 72 - protein, 20 aa, Artificial Sequence
  Synthetic peptide
TLDIQNKKITEVALMGHLSC

SEQ ID NO: 73 - protein, 20 aa, Artificial Sequence
  Synthetic peptide
GHLSCDTKEERKIKGVISIP

SEQ ID NO: 74 - protein, 20 aa, Artificial Sequence
  Synthetic peptide
VISIPRLQAEARSEILAHWS

SEQ ID NO: 75 - protein, 20 aa, Artificial Sequence
  Synthetic peptide
LAHWSPAKLLLQMDSSATAY

SEQ ID NO: 76 - protein, 20 aa, Artificial Sequence
  Synthetic peptide
SATAYGSTVSKRVAWHYDEE

SEQ ID NO: 77 - protein, 20 aa, Artificial Sequence
  Synthetic peptide
HYDEEKIEFEWNTGTNVDTK

SEQ ID NO: 78 - protein, 20 aa, Artificial Sequence
  Synthetic peptide
NVDTKKMTSNFPVDLSDYPK

SEQ ID NO: 79 - protein, 20 aa, Artificial Sequence
  Synthetic peptide
SDYPKSLHMYANRLLDHRVP

SEQ ID NO: 80 - protein, 20 aa, Artificial Sequence
  Synthetic peptide
DHRVPETDMTFRHVGSKLIV

SEQ ID NO: 81 - protein, 20 aa, Artificial Sequence
  Synthetic peptide
SKLIVAMSSWLQKASGSLPY

SEQ ID NO: 82 - protein, 20 aa, Artificial Sequence
  Synthetic peptide
GSLPYTQTLQDHLNSLKEFN

SEQ ID NO: 83 - protein, 20 aa, Artificial Sequence
  Synthetic peptide
LKEFNLQNMGLPDFHIPENL

SEQ ID NO: 84 - protein, 20 aa, Artificial Sequence
  Synthetic peptide
IPENLFLKSDGRVKYTLNKN

SEQ ID NO: 85 - protein, 20 aa, Artificial Sequence
  Synthetic peptide
TLNKNSLKIEIPLPFGGKSS

SEQ ID NO: 86 - protein, 20 aa, Artificial Sequence
  Synthetic peptide
GGKSSRDLKMLETVRTPALH

SEQ ID NO: 87 - protein, 20 aa, Artificial Sequence
  Synthetic peptide
TPALHFKSVGFHLPSREFQV

SEQ ID NO: 88 - protein, 20 aa, Artificial Sequence
  Synthetic peptide
REFQVPTFTIPKLYQLQVPL

SEQ ID NO: 89 - protein, 20 aa, Artificial Sequence
  Synthetic peptide
LQVPLLGVLDLSTNVYSNLY

SEQ ID NO: 90 - protein, 20 aa, Artificial Sequence
  Synthetic peptide
YSNLYNWSASYSGGNTSTDH

SEQ ID NO: 91 - protein, 20 aa, Artificial Sequence
  Synthetic peptide
TSTDHFSLRARYHMKADSVV

SEQ ID NO: 92 - protein, 20 aa, Artificial Sequence
  Synthetic peptide
ADSVVDLLSYNVQGSGETTY

SEQ ID NO: 93 - protein, 20 aa, Artificial Sequence
  Synthetic peptide
GETTYDHKNTFTLSCDGSLR

SEQ ID NO: 94 - protein, 20 aa, Artificial Sequence
  Synthetic peptide
DGSLRHKFLDSNIKFSHVEK

SEQ ID NO: 95 - protein, 20 aa, Artificial Sequence
  Synthetic peptide
SHVEKLGNNPVSKGLLIFDA

SEQ ID NO: 96 - protein, 20 aa, Artificial Sequence
  Synthetic peptide
LIFDASSSWGPQMSASVHLD

SEQ ID NO: 97 - protein, 20 aa, Artificial Sequence
  Synthetic peptide
SVHLDSKKKQHLFVKEVKID

SEQ ID NO: 98 - protein, 20 aa, Artificial Sequence
  Synthetic peptide
EVKIDGQFRVSSFYAKGTYG

SEQ ID NO: 99 - protein, 20 aa, Artificial Sequence
  Synthetic peptide
KGTYGLSCQRDPNTGRLNGE

SEQ ID NO: 100 - protein, 20 aa, Artificial Sequence
  Synthetic peptide
RLNGESNLRFNSSYLQGTNQ

SEQ ID NO: 101 - protein, 20 aa, Artificial Sequence
  Synthetic peptide
QGTNQITGRYEDGTLSLTST

SEQ ID NO: 102 - protein, 20 aa, Artificial Sequence
  Synthetic peptide
SLTSTSDLQSGIIKNTASLK

SEQ ID NO: 103 - protein, 20 aa, Artificial Sequence
  Synthetic peptide
TASLKYENYELTLKSDTNGK

SEQ ID NO: 104 - protein, 20 aa, Artificial Sequence
  Synthetic peptide
DTNGKYKNFATSNKMDMTFS

SEQ ID NO: 105 - protein, 20 aa, Artificial Sequence
  Synthetic peptide
DMTFSKQNALLRSEYQADYE

SEQ ID NO: 106 - protein, 20 aa, Artificial Sequence
  Synthetic peptide
QADYESLRFFSLLSGSLNSH

SEQ ID NO: 107 - protein, 20 aa, Artificial Sequence
  Synthetic peptide
SLNSHGLELNADILGTDKIN

SEQ ID NO: 108 - protein, 20 aa, Artificial Sequence
  Synthetic peptide
TDKINSGAHKATLRIGQDGI

SEQ ID NO: 109 - protein, 20 aa, Artificial Sequence
  Synthetic peptide
GQDGISTSATTNLKCSLLVL

SEQ ID NO: 110 - protein, 20 aa, Artificial Sequence
  Synthetic peptide
SLLVLENELNAELGLSGASM

SEQ ID NO: 111 - protein, 20 aa, Artificial Sequence
  Synthetic peptide
SGASMKLTTNGRFREHNAKF

SEQ ID NO: 112 - protein, 20 aa, Artificial Sequence
  Synthetic peptide
HNAKFSLDGKAALTELSLGS

SEQ ID NO: 113 - protein, 20 aa, Artificial Sequence
  Synthetic peptide
LSLGSAYQAMILGVDSKNIF

SEQ ID NO: 114 - protein, 20 aa, Artificial Sequence
  Synthetic peptide
SKNIFNFKVSQEGLKLSNDM

SEQ ID NO: 115 - protein, 20 aa, Artificial Sequence
  Synthetic peptide
LSNDMMGSYAEMKFDHTNSL

SEQ ID NO: 116 - protein, 20 aa, Artificial Sequence
  Synthetic peptide
HTNSLNIAGLSLDFSSKLDN

SEQ ID NO: 117 - protein, 20 aa, Artificial Sequence
  Synthetic peptide
SKLDNIYSSDKFYKQTVNLQ

SEQ ID NO: 118 - protein, 20 aa, Artificial Sequence
  Synthetic peptide
TVNLQLQPYSLVTTLNSDLK

SEQ ID NO: 119 - protein, 20 aa, Artificial Sequence
  Synthetic peptide
NSDLKYNALDLTNNGKLRLE

SEQ ID NO: 120 - protein, 20 aa, Artificial Sequence
  Synthetic peptide
KLRLEPLKLHVAGNLKGAYQ

SEQ ID NO: 121 - protein, 20 aa, Artificial Sequence
  Synthetic peptide
KGAYQNNEIKHIYAISSAAL

SEQ ID NO: 122 - protein, 20 aa, Artificial Sequence
  Synthetic peptide
SSAALSASYKADTVAKVQGV

SEQ ID NO: 123 - protein, 20 aa, Artificial Sequence
  Synthetic peptide
KVQGVEFSHRLNTDIAGLAS

SEQ ID NO: 124 - protein, 20 aa, Artificial Sequence
  Synthetic peptide
AGLASAIDMSTNYNSDSLHF

SEQ ID NO: 125 - protein, 20 aa, Artificial Sequence
  Synthetic peptide
DSLHFSNVFRSVMAPFTMTI

SEQ ID NO: 126 - protein, 20 aa, Artificial Sequence
  Synthetic peptide
FTMTIDAHTNGNGKLALWGE

SEQ ID NO: 127 - protein, 20 aa, Artificial Sequence
  Synthetic peptide
ALWGEHTGQLYSKFLLKAEP

SEQ ID NO: 128 - protein, 20 aa, Artificial Sequence
  Synthetic peptide
LKAEPLAFTFSHDYKGSTSH

SEQ ID NO: 129 - protein, 20 aa, Artificial Sequence
  Synthetic peptide
GSTSHHLVSRKSISAALEHK

SEQ ID NO: 130 - protein, 20 aa, Artificial Sequence
  Synthetic peptide
ALEHKVSALLTPAEQTGTWK

SEQ ID NO: 131 - protein, 20 aa, Artificial Sequence
  Synthetic peptide
TGTWKLKTQFNNNEYSQDLD

SEQ ID NO: 132 - protein, 20 aa, Artificial Sequence
  Synthetic peptide
SQDLDAYNTKDKIGVELTGR

SEQ ID NO: 133 - protein, 20 aa, Artificial Sequence
  Synthetic peptide
ELTGRTLADLTLLDSPIKVP

SEQ ID NO: 134 - protein, 20 aa, Artificial Sequence
  Synthetic peptide
PIKVPLLLSEPINIIDALEM

SEQ ID NO: 135 - protein, 20 aa, Artificial Sequence
  Synthetic peptide
DALEMRDAVEKPQEFTIVAF

SEQ ID NO: 136 - protein, 20 aa, Artificial Sequence
  Synthetic peptide
TIVAFVKYDKNQDVHSINLP

SEQ ID NO: 137 - protein, 20 aa, Artificial Sequence
  Synthetic peptide
SINLPFFETLQEYFERNRQT

SEQ ID NO: 138 - protein, 20 aa, Artificial Sequence
  Synthetic peptide
RNRQTIIVVVENVQRNLKHI

SEQ ID NO: 139 - protein, 20 aa, Artificial Sequence
  Synthetic peptide
NLKHINIDQFVRKYRAALGK

SEQ ID NO: 140 - protein, 20 aa, Artificial Sequence
  Synthetic peptide
AALGKLPQQANDYLNSFNWE

SEQ ID NO: 141 - protein, 20 aa, Artificial Sequence
  Synthetic peptide
SFNWERQVSHAKEKLTALTK

SEQ ID NO: 142 - protein, 20 aa, Artificial Sequence
  Synthetic peptide
TALTKKYRITENDIQIALDD

SEQ ID NO: 143 - protein, 20 aa, Artificial Sequence
  Synthetic peptide
IALDDAKINFNEKLSQLQTY

SEQ ID NO: 144 - protein, 20 aa, Artificial Sequence
  Synthetic peptide
QLQTYMIQFDQYIKDSYDLH

SEQ ID NO: 145 - protein, 20 aa, Artificial Sequence
  Synthetic peptide
SYDLHDLKIAIANIIDEIIE

SEQ ID NO: 146 - protein, 20 aa, Artificial Sequence
  Synthetic peptide
DEIIEKLKSLDEHYHIRVNL

SEQ ID NO: 147 - protein, 20 aa, Artificial Sequence
  Synthetic peptide
IRVNLVKTIHDLHLFIENID

SEQ ID NO: 148 - protein, 20 aa, Artificial Sequence
  Synthetic peptide
IENIDFNKSGSSTASWIQNV

SEQ ID NO: 149 - protein, 20 aa, Artificial Sequence
  Synthetic peptide
WIQNVDTKYQIRIQIQEKLQ

SEQ ID NO: 150 - protein, 20 aa, Artificial Sequence
  Synthetic peptide
QEKLQQLKRHIQNIDIQHLA

SEQ ID NO: 151 - protein, 20 aa, Artificial Sequence
  Synthetic peptide
IQHLAGKLKQHIEAIDVRVL

SEQ ID NO: 152 - protein, 20 aa, Artificial Sequence
  Synthetic peptide
DVRVLLDQLGTTISFERIND

SEQ ID NO: 153 - protein, 20 aa, Artificial Sequence
  Synthetic peptide
ERINDVLEHVKHFVINLIGD

SEQ ID NO: 154 - protein, 20 aa, Artificial Sequence
  Synthetic peptide
NLIGDFEVAEKINAFRAKVH

SEQ ID NO: 155 - protein, 20 aa, Artificial Sequence
  Synthetic peptide
RAKVHELIERYEVDQQIQVL

SEQ ID NO: 156 - protein, 20 aa, Artificial Sequence
  Synthetic peptide
QIQVLMDKLVELTHQYKLKE

SEQ ID NO: 157 - protein, 20 aa, Artificial Sequence
  Synthetic peptide
YKLKETIQKLSNVLQQVKIK

SEQ ID NO: 158 - protein, 20 aa, Artificial Sequence
  Synthetic peptide
QVKIKDYFEKLVGFIDDAVK

SEQ ID NO: 159 - protein, 20 aa, Artificial Sequence
  Synthetic peptide
DDAVKKLNELSFKTFIEDVN

SEQ ID NO: 160 - protein, 20 aa, Artificial Sequence
  Synthetic peptide
IEDVNKFLDMLIKKLKSFDY

SEQ ID NO: 161 - protein, 20 aa, Artificial Sequence
  Synthetic peptide
KSFDYHQFVDETNDKIREVT

SEQ ID NO: 162 - protein, 20 aa, Artificial Sequence
  Synthetic peptide
IREVTQRLNGEIQALELPQK

SEQ ID NO: 163 - protein, 20 aa, Artificial Sequence
  Synthetic peptide
ELPQKAEALKLFLEETKATV

SEQ ID NO: 164 - protein, 20 aa, Artificial Sequence
  Synthetic peptide
TKATVAVYLESLQDTKITLI

SEQ ID NO: 165 - protein, 20 aa, Artificial Sequence
  Synthetic peptide
KITLIINWLQEALSSASLAH

SEQ ID NO: 166 - protein, 20 aa, Artificial Sequence
  Synthetic peptide
ASLAHMKAKFRETLEDTRDR

SEQ ID NO: 167 - protein, 20 aa, Artificial Sequence
  Synthetic peptide
DTRDRMYQMDIQQELQRYLS

SEQ ID NO: 168 - protein, 20 aa, Artificial Sequence
  Synthetic peptide
QRYLSLVGQVYSTLVTYISD

SEQ ID NO: 169 - protein, 20 aa, Artificial Sequence
  Synthetic peptide
TYISDWWTLAAKNLTDFAEQ

SEQ ID NO: 170 - protein, 20 aa, Artificial Sequence
  Synthetic peptide
DFAEQYSIQDWAKRMKALVE

SEQ ID NO: 171 - protein, 20 aa, Artificial Sequence
  Synthetic peptide
KALVEQGFTVPEIKTILGTM

SEQ ID NO: 172 - protein, 20 aa, Artificial Sequence
  Synthetic peptide
ILGTMPAFEVSLQALQKATF

SEQ ID NO: 173 - protein, 20 aa, Artificial Sequence
  Synthetic peptide
QKATFQTPDFIVPLTDLRIP

SEQ ID NO: 174 - protein, 20 aa, Artificial Sequence
  Synthetic peptide
DLRIPSVQINFKDLKNIKIP

SEQ ID NO: 175 - protein, 20 aa, Artificial Sequence
  Synthetic peptide
NIKIPSRFSTPEFTILNTFH

SEQ ID NO: 176 - protein, 20 aa, Artificial Sequence
  Synthetic peptide
LNTFHIPSFTIDFVEMKVKI

SEQ ID NO: 177 - protein, 20 aa, Artificial Sequence
  Synthetic peptide
MKVKIIRTIDQMQNSELQWP

SEQ ID NO: 178 - protein, 20 aa, Artificial Sequence
  Synthetic peptide
ELQWPVPDIYLRDLKVEDIP

SEQ ID NO: 179 - protein, 20 aa, Artificial Sequence
  Synthetic peptide
VEDIPLARITLPDFRLPEIA

SEQ ID NO: 180 - protein, 20 aa, Artificial Sequence
  Synthetic peptide
LPEIAIPEFIIPTLNLNDFQ

SEQ ID NO: 181 - protein, 20 aa, Artificial Sequence
  Synthetic peptide
LNDFQVPDLHIPEFQLPHIS

SEQ ID NO: 182 - protein, 20 aa, Artificial Sequence
  Synthetic peptide
LPHISHTIEVPTFGKLYSIL

SEQ ID NO: 183 - protein, 20 aa, Artificial Sequence
  Synthetic peptide
LYSILKIQSPLFTLDANADI

SEQ ID NO: 184 - protein, 20 aa, Artificial Sequence
  Synthetic peptide
ANADIGNGTTSANEAGIAAS

SEQ ID NO: 185 - protein, 20 aa, Artificial Sequence
  Synthetic peptide
GIAASITAKGESKLEVLNFD

SEQ ID NO: 186 - protein, 20 aa, Artificial Sequence
  Synthetic peptide
VLNFDFQANAQLSNPKINPL

SEQ ID NO: 187 - protein, 20 aa, Artificial Sequence
  Synthetic peptide
KINPLALKESVKFSSKYLRT

SEQ ID NO: 188 - protein, 20 aa, Artificial Sequence
  Synthetic peptide
KYLRTEHGSEMLFFGNAIEG

SEQ ID NO: 189 - protein, 20 aa, Artificial Sequence
  Synthetic peptide
NAIEGKSNTVASLHTEKNTL

SEQ ID NO: 190 - protein, 20 aa, Artificial Sequence
  Synthetic peptide
EKNTLELSNGVIVKINNQLT

SEQ ID NO: 191 - protein, 20 aa, Artificial Sequence
  Synthetic peptide
NNQLTLDSNTKYFHKLNIPK

SEQ ID NO: 192 - protein, 20 aa, Artificial Sequence
  Synthetic peptide
LNIPKLDFSSQADLRNEIKT

SEQ ID NO: 193 - protein, 20 aa, Artificial Sequence
  Synthetic peptide
NEIKTLLKAGHIAWTSSGKG

SEQ ID NO: 194 - protein, 20 aa, Artificial Sequence
  Synthetic peptide
SSGKGSWKWACPRFSDEGTH

SEQ ID NO: 195 - protein, 20 aa, Artificial Sequence
  Synthetic peptide
DEGTHESQISFTIEGPLTSF

SEQ ID NO: 196 - protein, 20 aa, Artificial Sequence
  Synthetic peptide
PLTSFGLSNKINSKHLRVNQ

SEQ ID NO: 197 - protein, 20 aa, Artificial Sequence
  Synthetic peptide
LRVNQNLVYESGSLNFSKLE

SEQ ID NO: 198 - protein, 20 aa, Artificial Sequence
  Synthetic peptide
FSKLEIQSQVDSQHVGHSVL

SEQ ID NO: 199 - protein, 20 aa, Artificial Sequence
  Synthetic peptide
GHSVLTAKGMALFGEGKAEF

SEQ ID NO: 200 - protein, 20 aa, Artificial Sequence
  Synthetic peptide
GKAEFTGRHDAHLNGKVIGT

SEQ ID NO: 201 - protein, 20 aa, Artificial Sequence
  Synthetic peptide
KVIGTLKNSLFFSAQPFEIT

SEQ ID NO: 202 - protein, 20 aa, Artificial Sequence
  Synthetic peptide
PFEITASTNNEGNLKVRFPL

SEQ ID NO: 203 - protein, 20 aa, Artificial Sequence
  Synthetic peptide
VRFPLRLTGKIDFLNNYALF

SEQ ID NO: 204 - protein, 20 aa, Artificial Sequence
  Synthetic peptide
NYALFLSPSAQQASWQVSAR

SEQ ID NO: 205 - protein, 20 aa, Artificial Sequence
  Synthetic peptide
QVSARFNQYKYNQNFSAGNN

SEQ ID NO: 206 - protein, 20 aa, Artificial Sequence
  Synthetic peptide
SAGNNENIMEAHVGINGEAN

SEQ ID NO: 207 - protein, 20 aa, Artificial Sequence
  Synthetic peptide
NGEANLDFLNIPLTIPEMRL

SEQ ID NO: 208 - protein, 20 aa, Artificial Sequence
  Synthetic peptide
PEMRLPYTIITTPPLKDFSL

SEQ ID NO: 209 - protein, 20 aa, Artificial Sequence
  Synthetic peptide
KDFSLWEKTGLKEFLKTTKQ

SEQ ID NO: 210 - protein, 20 aa, Artificial Sequence
  Synthetic peptide
KTTKQSFDLSVKAQYKKNKH

SEQ ID NO: 211 - protein, 20 aa, Artificial Sequence
  Synthetic peptide
KKNKHRHSITNPLAVLCEFI

SEQ ID NO: 212 - protein, 20 aa, Artificial Sequence
  Synthetic peptide
LCEFISQSIKSFDRHFEKNR

SEQ ID NO: 213 - protein, 20 aa, Artificial Sequence
  Synthetic peptide
FEKNRNNALDFVTKSYNETK

SEQ ID NO: 214 - protein, 20 aa, Artificial Sequence
  Synthetic peptide
YNETKIKFDKYKAEKSHDEL

SEQ ID NO: 215 - protein, 20 aa, Artificial Sequence
  Synthetic peptide
SHDELPRTFQIPGYTVPVVN

SEQ ID NO: 216 - protein, 20 aa, Artificial Sequence
  Synthetic peptide
VPVVNVEVSPFTIEMSAFGY

SEQ ID NO: 217 - protein, 20 aa, Artificial Sequence
  Synthetic peptide
SAFGYVFPKAVSMPSFSILG

SEQ ID NO: 218 - protein, 20 aa, Artificial Sequence
  Synthetic peptide
FSILGSDVRVPSYTLILPSL

SEQ ID NO: 219 - protein, 20 aa, Artificial Sequence
  Synthetic peptide
ILPSLELPVLHVPRNLKLSL

SEQ ID NO: 220 - protein, 20 aa, Artificial Sequence
  Synthetic peptide
LKLSLPHFKELCTISHIFIP

SEQ ID NO: 221 - protein, 20 aa, Artificial Sequence
  Synthetic peptide
HIFIPAMGNITYDFSFKSSV

SEQ ID NO: 222 - protein, 20 aa, Artificial Sequence
  Synthetic peptide
FKSSVITLNTNAELFNQSDI

SEQ ID NO: 223 - protein, 20 aa, Artificial Sequence
  Synthetic peptide
NQSDIVAHLLSSSSSVIDAL

SEQ ID NO: 224 - protein, 20 aa, Artificial Sequence
  Synthetic peptide
VIDALQYKLEGTTRLTRKRG

SEQ ID NO: 225 - protein, 20 aa, Artificial Sequence
  Synthetic peptide
TRKRGLKLATALSLSNKFVE

SEQ ID NO: 226 - protein, 20 aa, Artificial Sequence
  Synthetic peptide
NKFVEGSHNSTVSLTTKNME

SEQ ID NO: 227 - protein, 20 aa, Artificial Sequence
  Synthetic peptide
TKNMEVSVAKTTKAEIPILR

SEQ ID NO: 228 - protein, 20 aa, Artificial Sequence
  Synthetic peptide
IPILRMNFKQELNGNTKSKP

SEQ ID NO: 229 - protein, 20 aa, Artificial Sequence
  Synthetic peptide
TKSKPTVSSSMEFKYDFNSS

SEQ ID NO: 230 - protein, 20 aa, Artificial Sequence
  Synthetic peptide
DFNSSMLYSTAKGAVDHKLS

SEQ ID NO: 231 - protein, 20 aa, Artificial Sequence
  Synthetic peptide
DHKLSLESLTSYFSIESSTK

SEQ ID NO: 232 - protein, 20 aa, Artificial Sequence
  Synthetic peptide
ESSTKGDVKGSVLSREYSGT

SEQ ID NO: 233 - protein, 20 aa, Artificial Sequence
  Synthetic peptide
EYSGTIASEANTYLNSKSTR

SEQ ID NO: 234 - protein, 20 aa, Artificial Sequence
  Synthetic peptide
SKSTRSSVKLQGTSKIDDIW

SEQ ID NO: 235 - protein, 20 aa, Artificial Sequence
  Synthetic peptide
IDDIWNLEVKENFAGEATLQ

SEQ ID NO: 236 - protein, 20 aa, Artificial Sequence
  Synthetic peptide
EATLQRIYSLWEHSTKNHLQ

SEQ ID NO: 237 - protein, 20 aa, Artificial Sequence
  Synthetic peptide
KNHLQLEGLFFTNGEHTSKA

SEQ ID NO: 238 - protein, 20 aa, Artificial Sequence
  Synthetic peptide
HTSKATLELSPWQMSALVQV

SEQ ID NO: 239 - protein, 20 aa, Artificial Sequence
  Synthetic peptide
ALVQVHASQPSSFHDFPDLG

SEQ ID NO: 240 - protein, 20 aa, Artificial Sequence
  Synthetic peptide
FPDLGQEVALNANTKNQKIR

SEQ ID NO: 241 - protein, 20 aa, Artificial Sequence
  Synthetic peptide
NQKIRWKNEVRIHSGSFQSQ

SEQ ID NO: 242 - protein, 20 aa, Artificial Sequence
  Synthetic peptide
SFQSQVELSNDQEKAHLDIA

SEQ ID NO: 243 - protein, 20 aa, Artificial Sequence
  Synthetic peptide
HLDIAGSLEGHLRFLKNIIL

SEQ ID NO: 244 - protein, 20 aa, Artificial Sequence
  Synthetic peptide
KNIILPVYDKSLWDFLKLDV

SEQ ID NO: 245 - protein, 20 aa, Artificial Sequence
  Synthetic peptide
LKLDVTTSIGRRQHLRVSTA

SEQ ID NO: 246 - protein, 20 aa, Artificial Sequence
  Synthetic peptide
RVSTAFVYTKNPNGYSFSIP

SEQ ID NO: 247 - protein, 20 aa, Artificial Sequence
  Synthetic peptide
SFSIPVKVLADKFITPGLKL

SEQ ID NO: 248 - protein, 20 aa, Artificial Sequence
  Synthetic peptide
PGLKLNDLNSVLVMPTFHVP

SEQ ID NO: 249 - protein, 20 aa, Artificial Sequence
  Synthetic peptide
TFHVPFTDLQVPSCKLDFRE

SEQ ID NO: 250 - protein, 20 aa, Artificial Sequence
  Synthetic peptide
LDFREIQIYKKLRTSSFALN

SEQ ID NO: 251 - protein, 20 aa, Artificial Sequence
  Synthetic peptide
SFALNLPTLPEVKFPEVDVL

SEQ ID NO: 252 - protein, 20 aa, Artificial Sequence
  Synthetic peptide
EVDVLTKYSQPEDSLIPFFE

SEQ ID NO: 253 - protein, 20 aa, Artificial Sequence
  Synthetic peptide
IPFFEITVPESQLTVSQFTL

SEQ ID NO: 254 - protein, 20 aa, Artificial Sequence
  Synthetic peptide
SQFTLPKSVSDGIAALDLNA

SEQ ID NO: 255 - protein, 20 aa, Artificial Sequence
  Synthetic peptide
LDLNAVANKIADFELPTIIV

SEQ ID NO: 256 - protein, 20 aa, Artificial Sequence
  Synthetic peptide
PTIIVPEQTIEIPSIKFSVP

SEQ ID NO: 257 - protein, 20 aa, Artificial Sequence
  Synthetic peptide
KFSVPAGIVIPSFQALTARF

SEQ ID NO: 258 - protein, 20 aa, Artificial Sequence
  Synthetic peptide
LTARFEVDSPVYNATWSASL

SEQ ID NO: 259 - protein, 20 aa, Artificial Sequence
  Synthetic peptide
WSASLKNKADYVETVLDSTC

SEQ ID NO: 260 - protein, 20 aa, Artificial Sequence
  Synthetic peptide
LDSTCSSTVQFLEYELNVLG

SEQ ID NO: 261 - protein, 20 aa, Artificial Sequence
  Synthetic peptide
LNVLGTHKIEDGTLASKTKG

SEQ ID NO: 262 - protein, 20 aa, Artificial Sequence
  Synthetic peptide
SKTKGTLAHRDFSAEYEEDG

SEQ ID NO: 263 - protein, 20 aa, Artificial Sequence
  Synthetic peptide
YEEDGKFEGLQEWEGKAHLN

SEQ ID NO: 264 - protein, 20 aa, Artificial Sequence
  Synthetic peptide
KAHLNIKSPAFTDLHLRYQK

SEQ ID NO: 265 - protein, 20 aa, Artificial Sequence
  Synthetic peptide
LRYQKDKKGISTSAASPAVG

SEQ ID NO: 266 - protein, 20 aa, Artificial Sequence
  Synthetic peptide
SPAVGTVGMDMDEDDDFSKW

SEQ ID NO: 267 - protein, 20 aa, Artificial Sequence
  Synthetic peptide
DFSKWNFYYSPQSSPDKKLT

SEQ ID NO: 268 - protein, 20 aa, Artificial Sequence
  Synthetic peptide
DKKLTIFKTELRVRESDEET

SEQ ID NO: 269 - protein, 20 aa, Artificial Sequence
  Synthetic peptide
SDEETQIKVNWEEEAASGLL

SEQ ID NO: 270 - protein, 20 aa, Artificial Sequence
  Synthetic peptide
ASGLLTSLKDNVPKATGVLY

SEQ ID NO: 271 - protein, 20 aa, Artificial Sequence
  Synthetic peptide
TGVLYDYVNKYHWEHTGLTL

SEQ ID NO: 272 - protein, 20 aa, Artificial Sequence
  Synthetic peptide
TGLTLREVSSKLRRNLQNNA

SEQ ID NO: 273 - protein, 20 aa, Artificial Sequence
  Synthetic peptide
LQNNAEWVYQGAIRQIDDID

SEQ ID NO: 274 - protein, 20 aa, Artificial Sequence
  Synthetic peptide
IDDIDVRFQKAASGTTGTYQ

SEQ ID NO: 275 - protein, 20 aa, Artificial Sequence
  Synthetic peptide
TGTYQEWKDKAQNLYQELLT

SEQ ID NO: 276 - protein, 20 aa, Artificial Sequence
  Synthetic peptide
QELLTQEGQASFQGLKDNVF

SEQ ID NO: 277 - protein, 20 aa, Artificial Sequence
  Synthetic peptide
KDNVFDGLVRVTQKFHMKVK

SEQ ID NO: 278 - protein, 20 aa, Artificial Sequence
  Synthetic peptide
HMKVKHLIDSLIDFLNFPRF

SEQ ID NO: 279 - protein, 20 aa, Artificial Sequence
  Synthetic peptide
NFPRFQFPGKPGIYTREELC

SEQ ID NO: 280 - protein, 20 aa, Artificial Sequence
  Synthetic peptide
REELCTMFIREVGTVLSQVY

SEQ ID NO: 281 - protein, 20 aa, Artificial Sequence
  Synthetic peptide
LSQVYSKVHNGSEILFSYFQ

SEQ ID NO: 282 - protein, 20 aa, Artificial Sequence
  Synthetic peptide
FSYFQDLVITLPFELRKHKL

SEQ ID NO: 283 - protein, 20 aa, Artificial Sequence
  Synthetic peptide
RKHKLIDVISMYRELLKDLS

SEQ ID NO: 284 - protein, 20 aa, Artificial Sequence
  Synthetic peptide
LKDLSKEAQEVFKAIQSLKT

SEQ ID NO: 285 - protein, 20 aa, Artificial Sequence
  Synthetic peptide
QSLKTTEVLRNLQDLLQFIF

SEQ ID NO: 286 - protein, 20 aa, Artificial Sequence
  Synthetic peptide
LQFIFQLIEDNIKQLKEMKF

SEQ ID NO: 287 - protein, 20 aa, Artificial Sequence
  Synthetic peptide
KEMKFTYLINYIQDEINTIF

SEQ ID NO: 288 - protein, 20 aa, Artificial Sequence
  Synthetic peptide
INTIFNDYIPYVFKLLKENL

SEQ ID NO: 289 - protein, 20 aa, Artificial Sequence
  Synthetic peptide
LKENLCLNLHKFNEFIQNEL

SEQ ID NO: 290 - protein, 20 aa, Artificial Sequence
  Synthetic peptide
IQNELQEASQELQQIHQYIM

SEQ ID NO: 291 - protein, 20 aa, Artificial Sequence
  Synthetic peptide
HQYIMALREEYFDPSIVGWT

SEQ ID NO: 292 - protein, 20 aa, Artificial Sequence
  Synthetic peptide
IVGWTVKYYELEEKIVSLIK

SEQ ID NO: 293 - protein, 20 aa, Artificial Sequence
  Synthetic peptide
VSLIKNLLVALKDFHSEYIV

SEQ ID NO: 294 - protein, 20 aa, Artificial Sequence
  Synthetic peptide
SEYIVSASNFTSQLSSQVEQ

SEQ ID NO: 295 - protein, 20 aa, Artificial Sequence
  Synthetic peptide
SQVEQFLHRNIQEYLSILTD

SEQ ID NO: 296 - protein, 20 aa, Artificial Sequence
  Synthetic peptide
SILTDPDGKGKEKIAELSAT

SEQ ID NO: 297 - protein, 20 aa, Artificial Sequence
  Synthetic peptide
ELSATAQEIIKSQAIATKKI

SEQ ID NO: 298 - protein, 20 aa, Artificial Sequence
  Synthetic peptide
TKKIISDYHQQFRYKLQDFS

SEQ ID NO: 299 - protein, 20 aa, Artificial Sequence
  Synthetic peptide
LQDFSDQLSDYYEKFIAESK

SEQ ID NO: 300 - protein, 20 aa, Artificial Sequence
  Synthetic peptide
IAESKRLIDLSIQNYHTFLI

SEQ ID NO: 301 - protein, 20 aa, Artificial Sequence
  Synthetic peptide
HTFLIYITELLKKLQSTTVM

SEQ ID NO: 302 - protein, 20 aa, Artificial Sequence
  Synthetic peptide
STTVMNPYMKLAPGELTIIL